(19) United States
(12) Patent Application Publication
(10) Pub. No.: US 2022/0409217 A1
(43) Pub. Date: Dec. 29, 2022

(54) SURGICAL INSTRUMENTS AND METHODS OF USE

(71) Applicant: AZURMEDS Inc., Dallas, TX (US)

(72) Inventors: Jean-Marie Berger, Eden Prairie, MN (US); Pierric Deransart, Saint martin d'Urage (FR); Thomas Bradley Edwards, Houston, TX (US); Grant E. Garrigues, Hinsdale, IL (US); Armodios M. Hatzidakis, Denver, CO (US); Gregory P. Nicholson, Western Spring, IL (US); Thierry Manceau, Aix-les-Bains (FR); Alain Tornier, Saint-Ismier (FR)

(21) Appl. No.: 17/848,322

(22) Filed: Jun. 23, 2022

Related U.S. Application Data
(60) Provisional application No. 63/293,055, filed on Dec. 22, 2021.
(60) Provisional application No. 63/214,751, filed on Jun. 24, 2021.

(51) Int. Cl. A61B 17/16

(52) U.S. Cl. CPC A61B 17/1635

(57) ABSTRACT

The present disclosure describes surgical instruments for orthopedic surgery, the instruments including a variety of components including one or more of a first and second arm, a first and second side member, a first and second link member, a first and second jaw member, and/or a guide assembly.

### CROSS-REFERENCE TO RELATED APPLICATION

This application claims the benefit of and priority to U.S. Provisional Patent Application No. 63/214,751, filed on Jun. 24, 2021 and U.S. Provisional Application No. 63/293,055, filed Dec. 22, 2021, the entire disclosure of each is incorporated by reference herein.

### TECHNICAL FIELD

The present disclosure relates to the field of surgical instruments, and particularly to surgical instruments involved in orthopedic surgical procedures, such as bone block surgical procedures.

### BACKGROUND

Glenohumeral instability is a common musculoskeletal problem that leads to pain, decreased ability to participate in high-level activities, and a decrease in overall health. Shoulder instability treatment might require a surgical procedure. To determine the most appropriate procedure, the surgeon assesses if there any bony lesion to the shoulder bones. If significant bone deficiency is identified, a reconstruction of the bone stock using bone graft is required.

For anterior instability surgical treatment, the Latarjet procedure describes a coracoid bone block procedure. French surgeon Michel Latarjet first suggested that the horizontal limb of the coracoid process be fixed with a screw flush to the anteroinferior margin of the glenoid, making a horizontal incision through the fibers of the subscapularis.

Although the Latarjet procedure has been constantly improved since 1954, specific instrumentation has not been designed to effectively address the various steps of the procedure. For example, clinicians or surgeons are forced to improvise using general surgical instruments such as towel clamps to hold the coracoid process during harvesting and then cross hole drilling. This improvisation could result in non-parallel holes or, even worse, in breaking of the bone block which in turn could force abandonment of the procedure. Surgeons are also forced to “eyeball” the proper location of the coracoid graft, in an attempt to place it parallel and flush to the articulating surface of the glenoid.

Accordingly, there is a need for improved instrumentation to be used during orthopedic surgical procedures, such as a Latarjet procedure, with reduced susceptibility to coracoid breakage, increased graft fixation, and correct alignment of the coracoid to the articulating surface of the glenoid.

### SUMMARY

The present disclosure provides improved instrumentation designed for use in orthopedic surgical procedures, and particularly bone-grafting surgical procedures, such as a Latarjet procedure. The instruments described herein improve alignment of the bone graft, increase proper fixation, and/or reduce susceptibility to breakage.

Throughout the present disclosure the term “proximal” is intended to relate to the end of the instrument closest to the operator of the instrument (e.g., the clinician, surgeon, robot) and the term “distal” is intended to relate to the end of the instrument closest to the patient.

The surgical instruments described herein may include at least a first and second arm, a first and second side members, a v-shaped linkage, and a pair of jaw members. The first and second arms can be pivotably coupled to each other via a rotational joint. The first and second side members are positioned on opposite sides of the rotational joint. A proximal portion of the first side member pivotably connects a distal end portion of the second arm to the first arm proximal the rotational joint. A proximal portion of the second side member pivotably connects a distal end portion of the first arm to the second arm proximal the rotational joint. The v-shaped linkage pivotably connects the first arm to the second arm proximal the rotational joint.

In some embodiments, a first jaw member is coupled to a distal portion of the first side member, and a second jaw member is coupled to a distal portion of the second side member.

In some embodiments, the surgical instrument is configured as a surgical forceps. In some embodiments, the surgical instrument is configured to include a pistol-grip design.

In some embodiments, and particularly as a surgical forceps, a proximal end portion of the first arm may further include a passage defined therein behind a deflectable wall defined on an inner surface of the first arm.

In some embodiments, and particularly as a surgical forceps, the instrument may further include a ratchet assembly including a shaped first end portion pivotably coupled to a collar on the first proximal end portion of the first arm, the shaped first end portion including a first rib and optionally a second rib, and a locking arm extending therefrom, wherein the first rib extends into the first passage behind the deflectable wall, the second rib extends away from the first rib, and the locking arm includes a plurality of teeth on at least one surface thereof, at least one of the teeth configured to engage a pawl positioned on a second proximal end portion of the second arm to lock the first and second arms in a fixed position relative to each other.

In some embodiments, and particularly as a surgical forceps, the surgical instruments may further include a guide assembly. The guide assembly may be coupled to the rotational joint. The guide assembly may include one or more guide walls extending vertically away from the rotational joint the guide wall including one or more bores defined therethrough, the bores configured to receive drill bits or fasteners therethrough. In some embodiments, the height of the guide wall is greater than at least one, if not both, of the first and second jaw members.

In some embodiments, the first jaw member includes a first jaw body having an inner surface including treads, wherein the inner surface is concave and the second jaw member includes a second jaw body including a one or more tines on a proximal end portion thereof and convex flange on a distal end portion thereof.

In some embodiments, and particularly in a pistol-grip design, the surgical instruments described herein may include a clamp assembly, a handle portion, a gear assembly, and a guide assembly. In some embodiments, the surgical instruments described herein include a clamp assembly including: a first and second arm pivotably coupled to each other via a rotational joint; a first and second side member positioned on opposite sides of the rotational joint, a first proximal portion of the first side member pivotably connecting a second distal end portion of the second arm to the first arm proximal the rotational joint, and a second proximal portion of the second side member pivotably connecting a first distal end portion of the first arm to the second arm proximal the rotational joint; a v-shaped linkage pivotably connecting the first arm to the second arm proximal the rotational joint; a first jaw member coupled to a distal portion of the first side member; and, a second jaw member coupled to a distal portion of the second side member.

In some embodiments, and particularly in a pistol-grip design, the surgical instruments described herein include a handle portion including a support frame positioned on top of a handle body. The support frame may be configured to support the clamp assembly. The support frame including at least a base and a cover.

In some embodiments, and particularly in a pistol-grip design, the handle body extends along a longitudinal axis between a first free end portion and a second end portion connected to the support frame. The first free end portion of the handle body may further include a handle aperture defined therethrough and one or more finger grooves on a distal handle face thereof.

In some embodiments, and particularly in a pistol-grip design, the surgical instruments described herein may include a gear assembly operably coupling a handle portion to a clamp assembly.

In some embodiments, and particularly in a pistol-grip design, the second end portion of the handle body defines a handle cavity therein. The handle cavity may be configured to receive a first lower portion of the gear assembly therein, the first lower portion of the gear assembly including a first gear, a trigger, and a locking member. The first gear may be configured to rotate about a pivot point inside the handle cavity and includes a first set of teeth along an outer perimeter thereof.

In some embodiments, and particularly in a pistol-grip design, the trigger may be affixed to the first gear by a trigger arm, the trigger arm extending from the first gear inside the handle cavity, through a proximal handle slot defined on a proximal face of the handle body, to the trigger positioned outside the handle cavity. The trigger arm may be configured to pivot within the proximal handle slot to cause the first gear to pivot inside the handle cavity.

In some embodiments, and particularly in a pistol-grip design, the locking member may include a locking arm, a locking pawl, and a tab member. The locking arm extending between a first fixed end portion secured to a portion of the handle portion and a second free end portion including the locking pawl and the tab member on opposite sides thereof. The locking arm positioned within and extending along a length of a distal handle slot defined in a distal face of the handle body. The tab member extending out of the distal handle slot. The locking pawl may be configured to interact with the teeth of the first gear remain inside the handle cavity.

In some embodiments, and particularly in a pistol-grip design, the second end portion of the handle body further includes a handle window. The handle window may be configured to provide visibility of at least a portion of the first gear from outside the handle cavity.

In some embodiments, and particularly in a pistol-grip design, the gear assembly further includes a second upper portion received and/or maintained within the support frame. The second upper portion of the gear assembly including a second gear, a biasing member, and a clamp pin connecting the gear assembly to the clamp assembly.

In some embodiments, and particularly in a pistol-grip design, the second gear is a linear gear including a second set of teeth configured to interact with a portion of the first set of teeth of the first gear. The second gear being located within a gear channel of the base of the support frame. The second gear may be configured to slide side-to-side, relative to the first gear, within the gear channel.

In some embodiments, and particularly in a pistol-grip design, the biasing member is positioned proximal the second gear within the base of the support frame. The biasing member may be configured to force the second gear to slide distally within the gear channel. Alternatively, the biasing member may be configured to prevent the second gear from sliding distally within the gear channel.

In some embodiments, and particularly in a pistol-grip design, the biasing member is positioned distal the second gear within the base of the support frame. The biasing member may be configured to force the second gear to slide proximally within the gear channel. Alternatively, the biasing member may be configured to prevent the second gear from sliding proximally within the gear channel.

In some embodiments, the biasing member is a compression spring. In some embodiments, the biasing member is a tension spring.

In some embodiments, and particularly in a pistol-grip design, the clamping pin extends between a proximal end portion of the second gear to a pin slit defined in the cover of the support frame, with a proximal part of the v-shaped linkage positioned therebetween. The clamping pin may be configured to slide side-to-side within the pin slit forcing the proximal part of the v-shaped linkage to move proximally or distally thereby forcing the jaw members to open or close.

In some embodiments, and particularly in a pistol-grip design, the guide assembly includes an open guide channel, a guide carrier including a fixed first bore defined therethrough, and a removable guide insert including a removable second bore defined therethrough.

In some embodiments, the open guide channel is affixed atop the cover of the support frame and extends between the pin slit and the rotational joint.

In some embodiments, the guide carrier extends between a proximal part and a distal part, the proximal part affixed atop the cover of the support frame distal the rotational joint and the distal part extending away from the cover in a distal direction to a location prior to the first and second jaw members. The guide carrier further including a first guide shelf defining a first plane. The first guide shelf proximal a second guide shelf defining a second plane different than the first plane. The first guide shelf leading to a guide locking aperture configured to receive a guide locking post of the removable guide insert.

In some embodiments, the removable guide insert further includes the guide locking post located beneath the second bore. The guide locking post further includes a deflectable guide locking member on top of a distal part thereof. The guide locking post and the deflectable guide locking member may be configured to be received within the guide locking aperture to secure the removable guide insert to the guide carrier.

Methods of use and surgical kits including the surgical instruments described herein are also provided.

### BRIEF DESCRIPTION OF THE DRAWINGS

Various embodiments of the kits and/or components are described herein with reference to the drawings wherein:

FIG. 1A is perspective view of a surgical instrument as described in at least one embodiment herein;

FIGS. 1B and 1C are left and right side views of the surgical instrument of FIG. 1A.

FIG. 2A is a perspective view of a jaw member described in at least one embodiment herein;

FIG. 2B is a top view of the jaw member of FIG. 2A described in at least one embodiment herein;

FIG. 3A is a perspective view of a jaw member described in at least one embodiment herein;

FIG. 3B is an end view of the jaw member of FIG. 3A described in at least one embodiment herein;

FIG. 4 is a perspective view of a jaw member described in at least one embodiment herein;

FIGS. 5A and 5B are a perspective view and a top view, respectively, of a pair of jaw members as described in at least one embodiment herein;

FIG. 6A is a schematic cross-sectional view of a proximal end portion of a surgical instrument described in at least one embodiment herein;

FIG. 6B is a partial perspective view of a proximal end portion of a surgical instrument described in at least one embodiment herein;

FIG. 7 is a perspective view of a surgical instrument as described in at least one embodiment herein;

FIGS. 8A and 8B are perspective views of the distal end portions of the surgical instrument of FIG. 7 as described in at least one embodiment herein;

FIG. 9A is a side view of a jaw member described in at least one embodiment herein;

FIG. 9B is a perspective view of a jaw member described in at least one embodiment herein;

FIG. 10 is a top view of a surgical instrument as described in at least one embodiment herein;

FIG. 11 is a perspective view of a portion of a surgical procedure described in at least one embodiment herein;

FIG. 12A is a side view of a surgical instrument and graft as described in at least one embodiment herein;

FIG. 12B is a cross-sectional front view of the surgical instrument and graft of FIG. 12A as described in at least one embodiment herein;

FIG. 13A is a side view of a surgical instrument and graft as described in at least one embodiment herein;

FIG. 13B is a rear end view of the surgical instrument and graft of FIG. 13A as described in at least one embodiment herein;

FIG. 13C is a top view of the surgical instrument and graft of FIG. 13A as described in at least one embodiment herein;

FIG. 14A is a front view of a graft attached to tissue as described in at least one embodiment herein;

FIG. 14B is a cross-sectional side view of graft and tissue of FIG. 14A as described in at least one embodiment herein; and

FIG. 15 is a rear end view of a surgical instrument and graft as described in at least one embodiment herein.

FIG. 16A is a perspective view of a surgical instrument in a closed and/or clamping configuration as described in at least one embodiment herein;

FIGS. 16B and 16C are left and right side views of the surgical instrument of FIG. 16A as described in at least one embodiment herein.

FIG. 16D is a cross-sectional view of the surgical instrument of FIG. 16A (without the side arm and jaw members for clarity purposes) as described in at least one embodiment herein;

FIG. 16E is a top view of the surgical instrument of FIG. 16A as described in at least one embodiment herein;

FIG. 17A is a top view of a surgical instrument in an open and/or non-clamping configuration without a removable guide insert (with the jaw members depicted as removably attached) as described in at least one embodiment herein;

FIG. 17B is a rear view of the surgical instrument of FIG. 17A without a removable guide insert (without jaw members for clarity purposes) as described in at least one embodiment herein;

FIG. 17C is a side view of the surgical instrument of FIG. 17A and a removable guide insert (without the jaw members for clarity purposes) as described in at least one embodiment herein;

FIG. 17D is a cross-sectional view of the surgical instrument of FIG. 17C with the removable guide insert attached thereto (without the side arm and jaw members for clarity purposes) as described in at least one embodiment herein;

FIG. 18 is a cross-sectional view of a surgical instrument in an open or non-clamping configuration as described in at least one embodiment herein:

FIG. 19 is a perspective view of a jaw member described in at least one embodiment herein;

FIG. 20A is a perspective view of a guide insert described in at least one embodiment herein;

FIGS. 20B and 20C are a front and rear view, respectively, of a surgical instrument including the guide insert of FIG. 20A described in at least one embodiment herein;

FIG. 21A is a perspective view of a guide insert described in at least one embodiment herein;

FIG. 21B is a front view of a surgical instrument including the guide insert of FIG. 21A described in at least one embodiment herein;

FIGS. 22A and 22B are perspective views of a separate positioning tool as described in at least one embodiment herein;

FIG. 23 is a perspective view of a portion of a surgical procedure described in at least one embodiment herein;

FIG. 24 is a side view of a surgical instrument and graft as described in at least one embodiment herein;

FIG. 25A is a front view of a graft attached to tissue as described in at least one embodiment herein; and

FIG. 25B is a cross-sectional side view of graft and tissue of FIG. 25A as described in at least one embodiment herein.

### DETAILED DESCRIPTION

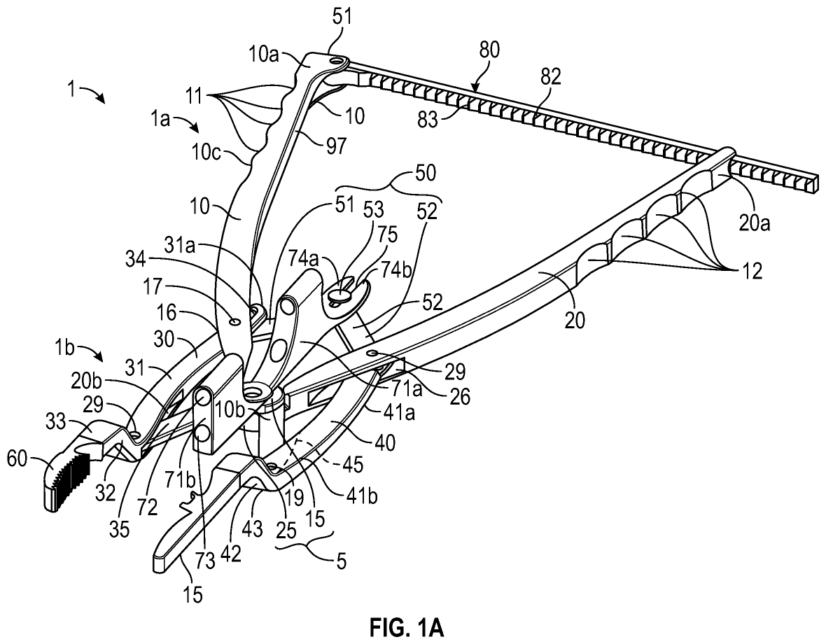

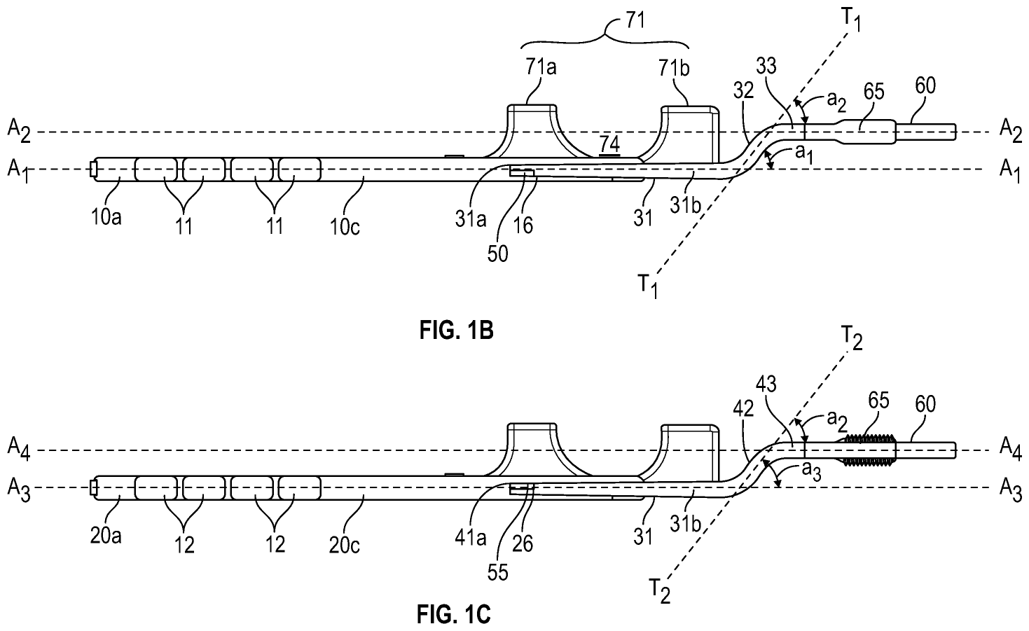

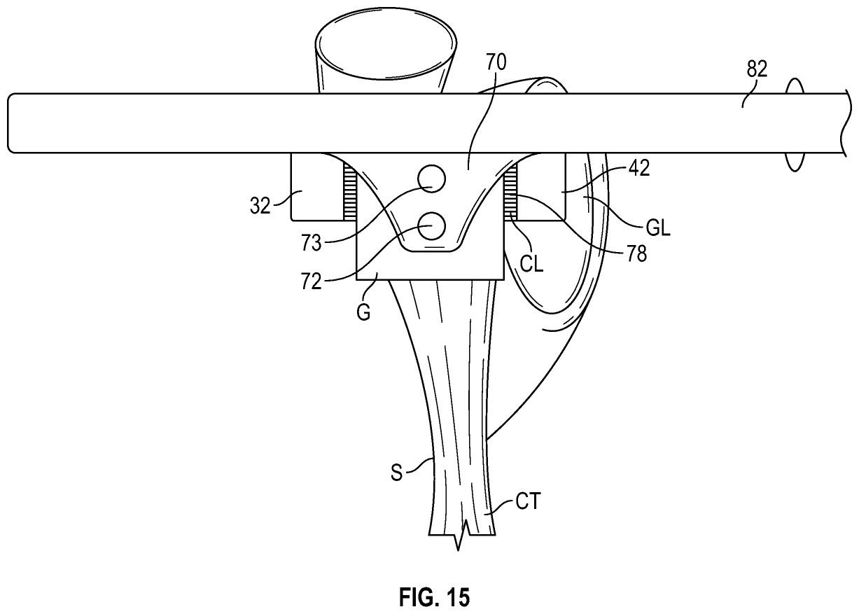

The present disclosure describes a surgical instrument, such as a clamping device, suitable for use in a surgical procedure. In some embodiments, the surgical instrument may be a clamping device such as surgical forceps (as shown at least in FIGS. 1A-1C). In some embodiments, the surgical instrument may be a clamping device such as a surgical clamping pistol (as shown at least in FIGS. 15A-15E). In some embodiments, the surgical instruments described herein are suitable for use in orthopedic surgical procedures, and particularly bone grafting procedures such as a Latarjet procedure.

I. Surgical Forceps Design

With reference to FIGS. 1A-1C, illustrated is an assembled and operable surgical instrument 1, and particularly a surgical clamp such as a surgical forceps 1, as described herein in at least one embodiment. The surgical instrument 1 includes a proximal end portion 1a designed to be closest to the user of the apparatus, such as a clinician or surgeon, and an opposite distal end portion 1b designed to be closest to the patient and/or farthest from the user.

FIG. 1A shows a perspective view of a surgical instrument 1 according to at least one embodiment described herein. The surgical instrument 1 may include a variety of components including, but not limited to, a first and second arm 10, 20, a first and second side member 30, 40, a first and second link member 50, 55, a first and second jaw members 60, 65, a guide assembly 70, and/or a ratchet assembly 80. The first and second arm 10, 20, the first and second side member 30, 40, the first and second link member 50, 55, and the first and second jaw members 60, 65 may form a clamp assembly of the surgical instrument 1. Each of the components of the surgical apparatus 1 may be constructed from any suitable sterilizable material including, but not limited to, stainless steel, silver, titanium, nitinol, and the like. Any suitable polymeric material may also be used including, but not limited to, polyether ether ketones (PEEK), polycarbonates, polyphenylsulfones (Rader)) and the like.

As shown in FIG. 1A, in some embodiments, the surgical instrument 1 may include a first arm 10 and a second arm 20 pivotably coupled to each other. The first and second arms 10, 20 are configured to pivot and/or rotate around a rotational joint 5 for opening and/or closing of the instrument 1, and more particularly, the opening and/or closing of a first and second jaw member 60, 65. The first and second arms 10, 20 overlap and/or cross over each other at the rotational joint 5.

In some embodiments, the rotational joint 5 may be positioned closer to the distal end portions 10b, 20b of the first and second arms 10, 20 than the proximal end portions 10a, 20a of the first and second arms 10, 20.

In addition to the first and second arms 10, 20, the surgical instruments 1 described herein further include first and second side members 30, 40 and a v-shaped linkage 50.

Each of the first and second side members 30, 40 includes: a proximal portion 31, 41 pivotably connected to both the first and second arms 10, 20; a distal portion 33, 43 configured to connect to a jaw member 60, 65; and an offset portion 32, 42 positioned therebetween.

As shown in FIG. 1A-1B, the proximal portion 31 of the first side member 30 extends between a first end part 31a pivotably coupled to a portion of the first arm 10 proximal the rotational joint 5 and a second end part 31b pivotably coupled to the distal end portion 20b of the second arm 20.

In some embodiments, the first end part 31a (of the proximal portion 31 of the first side member 30) includes a first member opening 34 defined therethrough, wherein at least some of the first end part 31a and/or the first member opening 34 is received within a first channel 16 defined through the first arm 10. The first channel 16 being positioned proximal to the rotational joint 5. A first proximal pin 17 passes perpendicularly through the first channel 16 of the first arm 10 and the first member opening 34 of the first side member 30 to pivotably couple the first side member 30 to the first arm 10.

In some embodiments, the second end part 31b (of the proximal portion 31 of the first side member 30) includes a first slot 35 defined therein, wherein at least some of the second distal end portion 20b of the second arm 20 is received within the first slot 35. A second distal pin 29 passes perpendicularly through the first slot 35 of the first side member 30 to pivotably couple the first side member 30 to the second distal end portion 20b of the second arm 20.

As further shown in FIGS. 1A-1B, the first offset portion 32 (of the first side member 30) is positioned between a generally planar first proximal portion 31 and a generally planar first distal portion 33. The first proximal portion 31 of the side member defines a first longitudinal axis A₁ and the first distal portion 33 defines a second longitudinal axis A₂ offset from the first longitudinal axis A₁. The first and second longitudinal axes A₁, A₂ being generally parallel to each other.

The first offset portion 32 defines a transverse axis T₁ extending at a first angle a₁ relative to longitudinal axis A₁ of the first proximal portion 31 and/or extending at a second angle a₂ relative to the longitudinal axis A₂ of the first distal portion 33. In some embodiments, the first and second angles a₁, a₂ may range from about 25-135°, individually. In some embodiments, the first and second angles a₁, a₂ may range from about 35-105°, individually. In some embodiments, the first and second angles a₁, a₂ may range from about 45-95°, individually. In some embodiments, the first and/or second angles a₁, a₂ may each be generally about 90°.

As further shown in FIGS. 1A-1B, the first distal portion 33 (of the first side member 30) is spaced distally and vertically from the second arm 20 by the first offset portion 32. The first distal portion 33 is coupled to the first jaw member 60. In some embodiments, the first distal portion 33 is non-pivotably coupled to the first jaw member 60.

As shown in FIGS. 1A and 1C, the proximal portion 41 of the second side member 40 extends between a first end part 41a pivotably coupled to a portion of the second arm 20 proximal the rotational joint 5 and a second end part 41b pivotably coupled to the first distal end portion 10b of the first arm 10.

In some embodiments, the first end part 41a (of the proximal portion 41 of the second side member 40) includes a second member opening (not shown) defined therethrough, wherein at least a some of the first end part 41a and/or the second member opening is received within a second channel 26 defined through the second arm 20. The second channel 26 being positioned proximal to the rotational joint 5. A second proximal pin 27 passes perpendicularly through the second channel 26 of the second arm 20 and the second member opening of the second side member 40 to pivotably couple the second side member 40 to the second arm 20.

In some embodiments, the second end part 41b (of the proximal portion 41 of the first side member 40) includes a second slot 45 defined therein, wherein at least some of the first distal end portion 10b of the first arm 10 is received within the second slot 45. A first distal pin 19 passes perpendicularly through the second slot 45 of the second side member 40 to pivotably couple the second side member 40 to the first distal end portion 10b of the first arm 10.

As further shown in FIGS. 1A and 1C, the second side member 40 includes a second offset portion 42 positioned between a generally planar second proximal portion 41 and a generally planar second distal portion 43. The second proximal portion 41 defines a third longitudinal axis A₃ and the second distal portion 43 defines a fourth longitudinal axis A₄ offset from the third longitudinal axis A₃. The third and fourth longitudinal axes A₃, A₄ being generally parallel to each other.

The second offset portion 42 of the second side member 40 defines a transverse axis T₂ extending at a third angle a₃ relative to the third longitudinal axis A₃ of the second proximal portion 41 and/or extending at a fourth angle a₄ relative to the fourth longitudinal axis A₄ of the second distal portion 43. In some embodiments, the third and fourth angles a₃, a₄ may range from about 25-135°, individually. In some embodiments, the third and fourth angles a₃, a₄ may range from about 35-105°, individually. In some embodiments, the third and fourth angles a₃, a₄ may range from about 45-95°, individually. In some embodiments, each of the third and fourth angles a₃, a₄ may be generally about 90°.

As further shown in FIGS. 1A and 1C, the first distal portion 43 (of the second side member 40) is spaced distally and vertically from the first arm 10 by the second offset portion 42. The second distal portion 43 is coupled to the first jaw member. In some embodiments, the first distal portion is non-pivotably coupled to the first jaw member 60.

The surgical instrument 1 may further include a v-shaped linkage 50 pivotably connecting the first arm 10 to the second arm 20 proximal to the rotational joint 5. The v-shaped linkage 50 includes a first link member 51 pivotably coupled to a second link member 52 via a link pivot joint on a proximal end portion of each of the first and second link members 51, 52.

A distal end portion of the first link member 51 is pivotably coupled to the first proximal pin 17 and received and maintained with the first channel 16. The distal end portion of the first link member 51 overlaps the first proximal portion 31a of the first side member 30 in the first channel 16.

A distal end portion of the second link member 52 is pivotably coupled to the second proximal pin 27 and received and maintained with the second channel 26. The distal end portion of the second link member 52 overlaps the second proximal portion 41a of the second side member 40 in the second channel 26.

A guide post 53 extends vertically from the link pivot joint in a direction perpendicular to the linkage 50. The guide post 53 is configured to interact with the guide assembly 70.

In some embodiments, guide assembly 70 of the surgical forceps 1 is secured to the rotational joint 5. However, unlike the first and second arms 10, 20, in some embodiments, the guide assembly 70 does not pivot relative to the rotational joint 5. In some embodiments, the guide assembly 70 is affixed on top of the rotational joint 5.

The guide assembly 70 may include a guide wall 71 extending vertically away from the rotational joint 5. The guide wall 71 may include one or more bores 72, 73 defined through the wall 71. The bores 72, 73 configured to receive drill bits and/or fasteners (e.g., pins, screws, bolts, nails, and the like) therethrough. The bores 72, 73 extend from a proximal end to a distal end of the guide wall 71. In some embodiments, the guide wall includes a first and second bore 72, 73 stacked one on top of the other.

As further depicted in FIG. 1A, in some embodiments, the guide wall 71 may include a proximal wall part 71a separated from a distal wall part 71b by a gap 74. Each of the wall parts 71a, 71b including the one or more bores 72, 73. In some embodiments, the gap 74 is semi-circular in design.

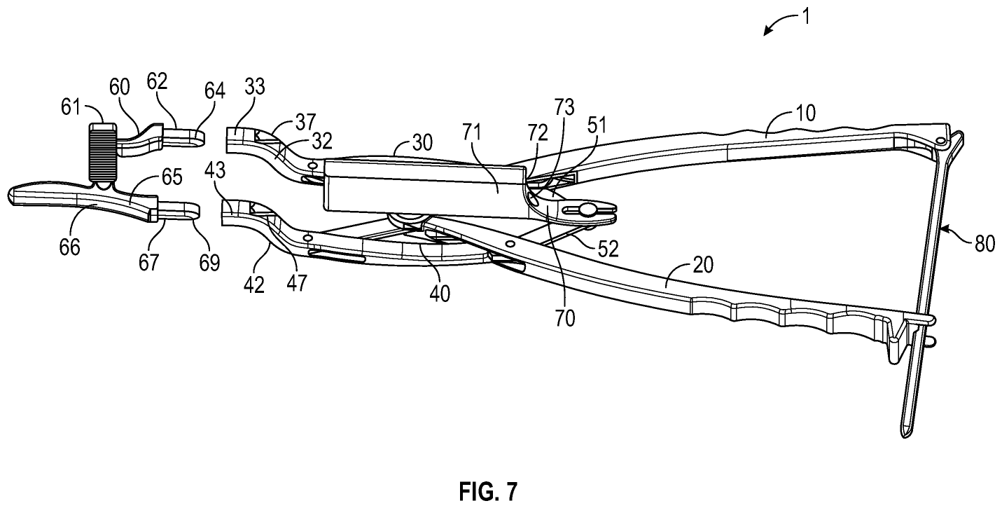

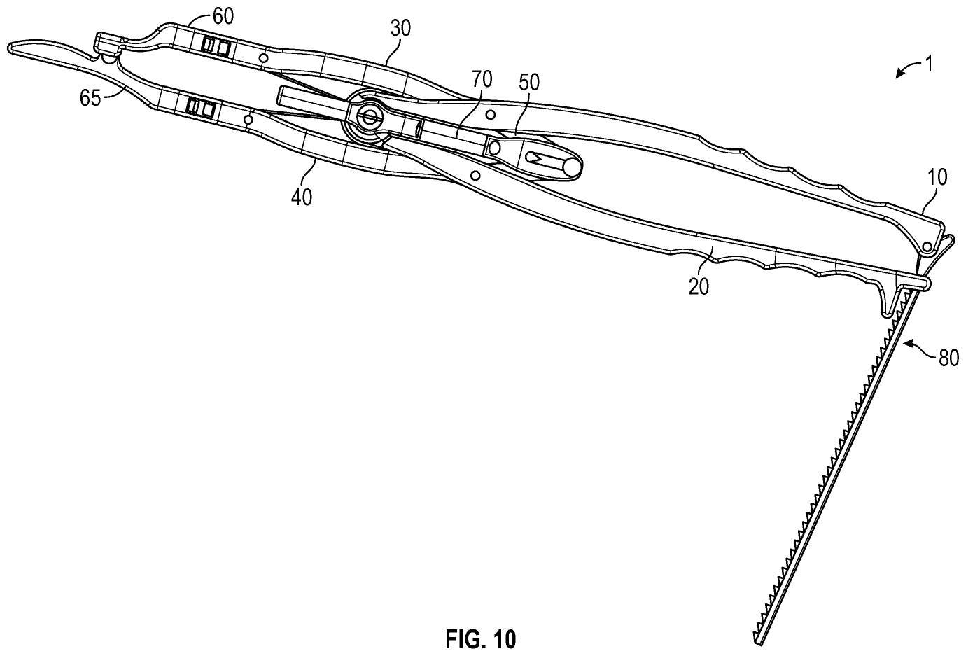

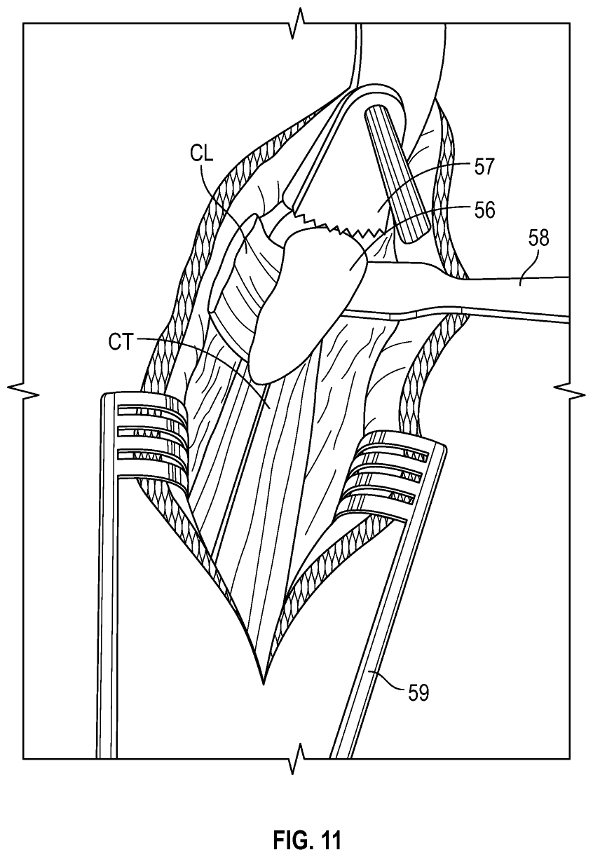

The guide assembly 70 may also include a pair of guide arms 74a, 74b separated by a guide slit 75 on a proximal portion thereof. The guide slit 75 is configured to interact with the guide post 53 of the linkage 50. Particularly, the guide post 53 is configured to be positioned within and/or slide within the guide slit 75 as the instrument 1 transitions between the open configuration or non-clamping (FIGS. 1A and 7) and the closed or clamping configuration (FIG. 10).

The guide post 53, the guide arms 74a, 74b, and the guide slit 75 are configured to maintain the guide assembly 70, and particularly the one or more bores 72, 73, in a fixed position relative to the pivoting parts of the instrument 1, such as the first and second arms 10, 20, the first and second side members 30, 40, and the linkage 50.

The guide post 53, the guide arms 74a, 74b, and the guide slit 75 are also configured to maintain the guide assembly 70, and particularly the one or more bores 72, 73, in a fixed position relative to the jaw members 60, 65. The guide assembly 70 is configured to be centered between the first and second jaw members 60, 65.

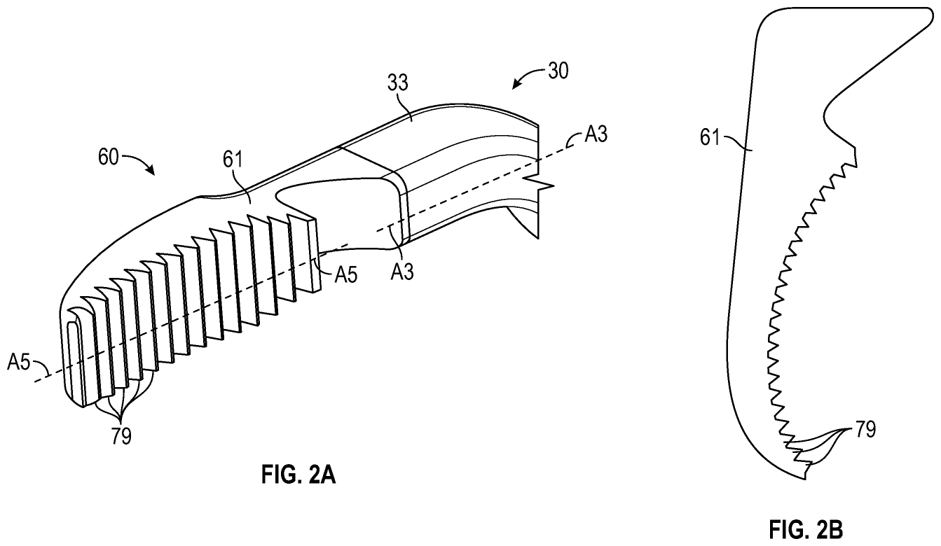

Turning to FIGS. 2A and 2B, in some embodiments, the first jaw member 60 is shown affixed to the first side member 30. As shown, the first jaw member 60 may include a first jaw body 61 defining a longitudinal axis A₅ coplanar and/or collinear with the longitudinal axis A₃ of the second distal planar end portion 33 of the first side member 30. A plurality of treads 79 may be formed on an inner surface of the first jaw member 60. In some embodiments, as best seen in FIG. 2B, the inner surface of the first jaw member 60 may define a concave surface along a length of the inner surface. In some embodiments, the plurality of treads 79 may extend generally perpendicular to the longitudinal axis A₅ of the first jaw member 60.

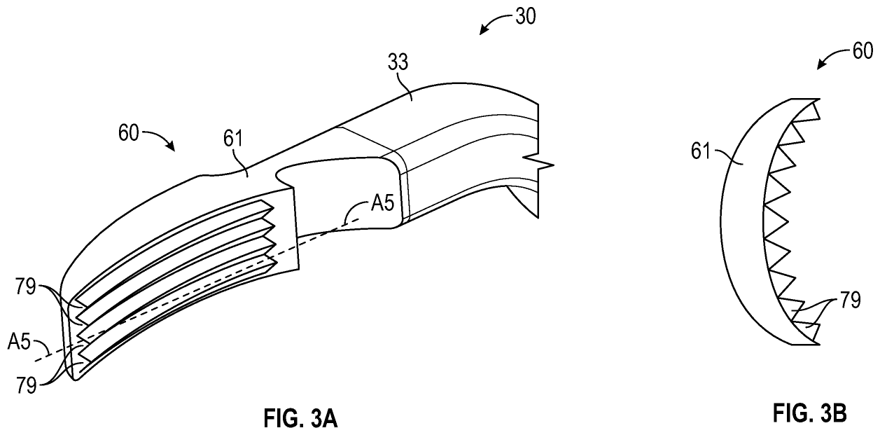

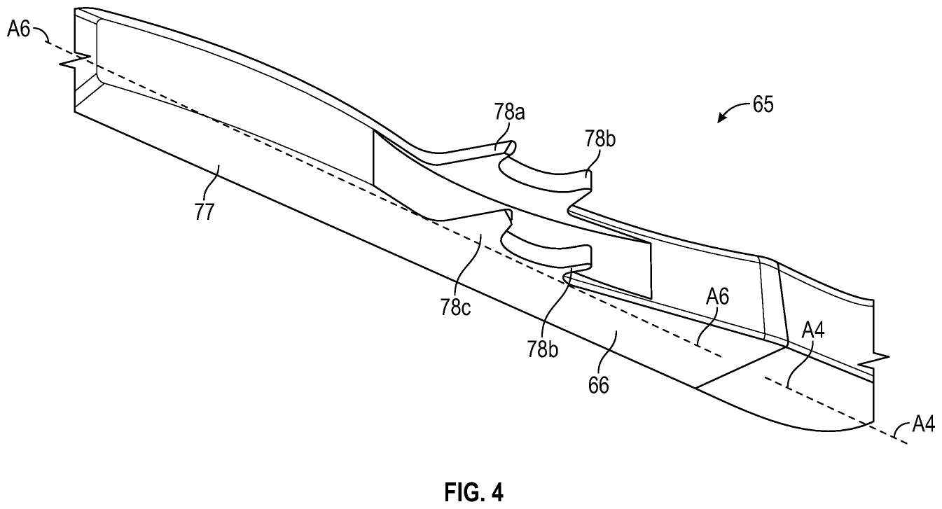

In some embodiments, as shown in FIGS. 3A and 3B, the first jaw member 60 may include a first jaw body 61 defining a longitudinal axis A₅ coplanar and/or collinear with the longitudinal axis A₃ of the second distal planar end portion 33 of the first side member 30. A plurality of treads 79 may be formed on an inner surface of the first jaw member 60. In some embodiments, as best seen in FIG. 3B, the inner surface of the first jaw member 60 may define a concave surface along a height of the inner surface.

In some embodiments, the plurality of treads 79 may extend generally along the longitudinal axis A₅ of the first jaw member 60.

As shown in FIGS. 1A and 4, the second jaw member 65 may include a second jaw body 66 defining a longitudinal axis A₆ coplanar with the longitudinal axis A₄ of the second distal end portion 43 of the second side member 40. The second jaw body 66 includes a proximal end portion 66a including at least one tine 78 and a distal end portion 66b. The second jaw member 65 may define a greater length than the first jaw member 60.

The at least one tine 78 may be formed on an inner surface of a proximal portion 66a of the second jaw body 66 (and/or jaw member 65). In some embodiments, the second jaw member 65 may include one or more pairs of tines 78a, 78b. In some embodiments, the second jaw member 65 may include a first pair of tines 78a, 78b and a second pair of tines 78c, 78d on opposite edges of the jaw body 66.

In some embodiments, the first and second pair of tines 78a, 78b, 78c, 78d may be separated by a concave surface 95 on the proximal end portion 66a. The concave surface providing additional space from the tips of each tine to accommodate a jagged or crenulated outer surface of a bone graft.

A flange 77, and particularly a convex and/or rounded flange 77, is formed on a surface of a distal end portion 66b of the second jaw body 66. The proximal end portion 66a of the second jaw member 65, and particularly the tines 78a-d, is configured to align with the treads 79 of the first jaw member 60 in the closed configuration.

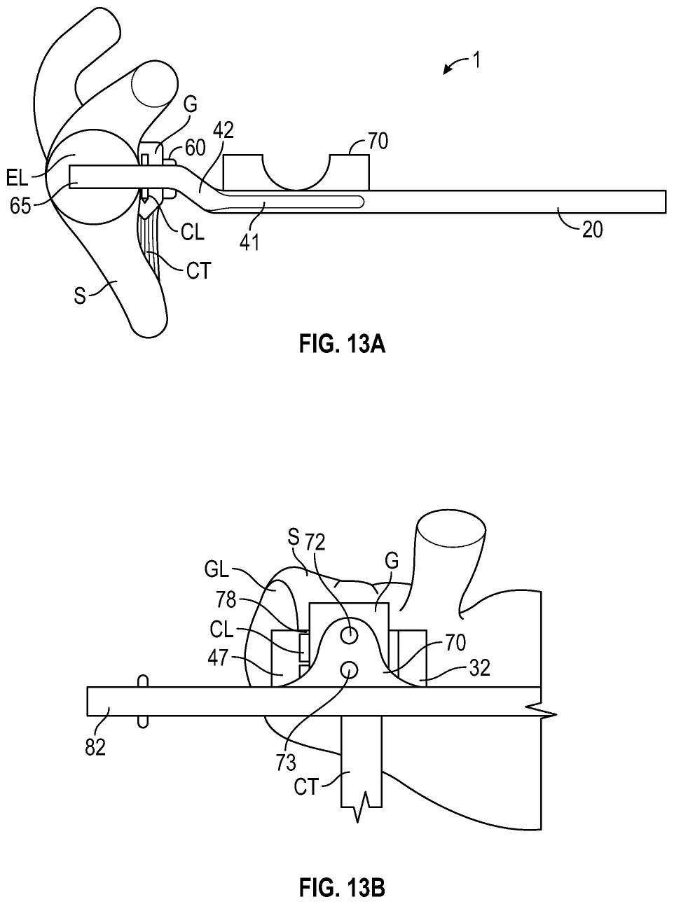

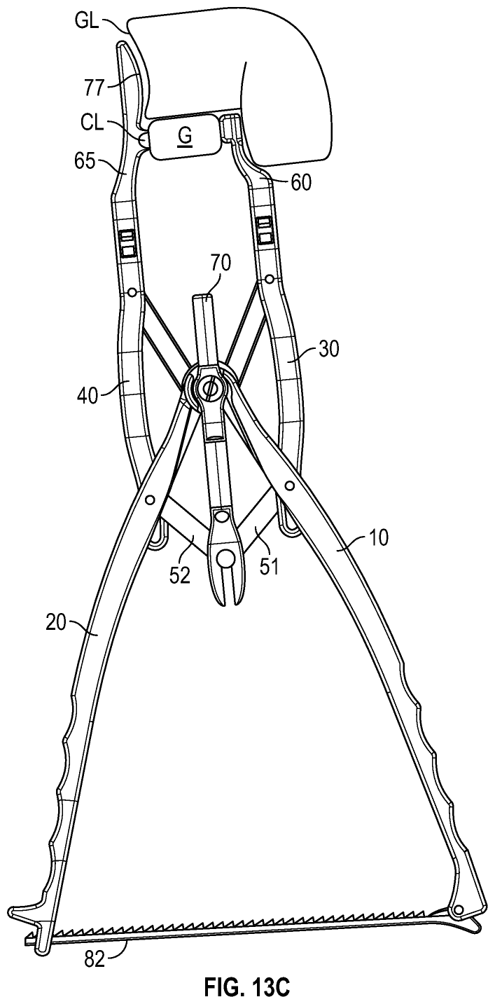

Since the flange and/or convex flange 77 extends beyond the one or more tines 78, the flange and/or convex flange 77 does not align with treads 79 and/or any portion of the first jaw member 60. As described in more detail hereinbelow, the flange and/or convex flange 77 is designed to act as a guide for interacting with an outer side surface of a bone to which a graft may be secured to. For example, in a shoulder surgery such as a Latarjet procedure, the convex flange 77 is configured to fit into a cavity defined in a side surface of the scapula when the bone graft is properly positioned on a front surface of the scapula (See FIG. 13C).

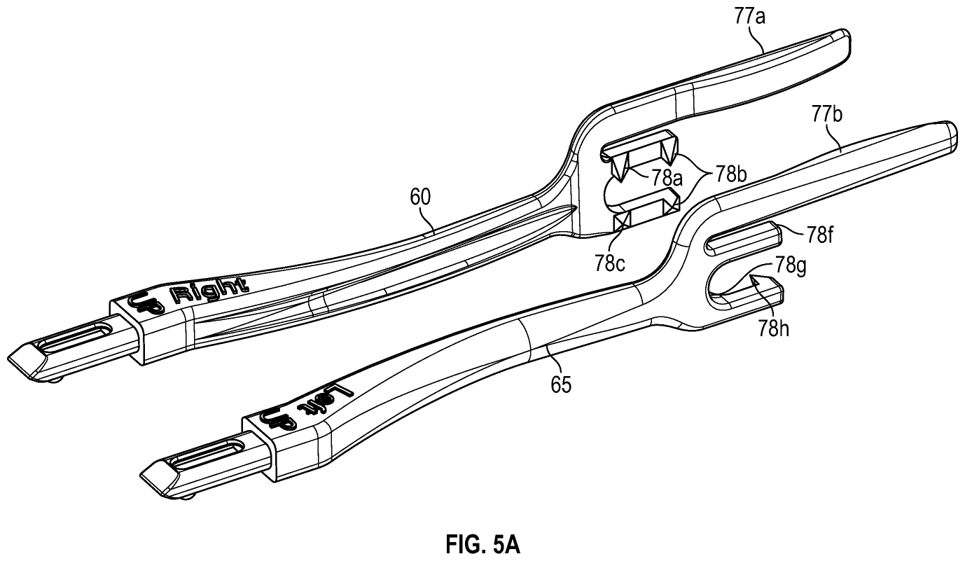

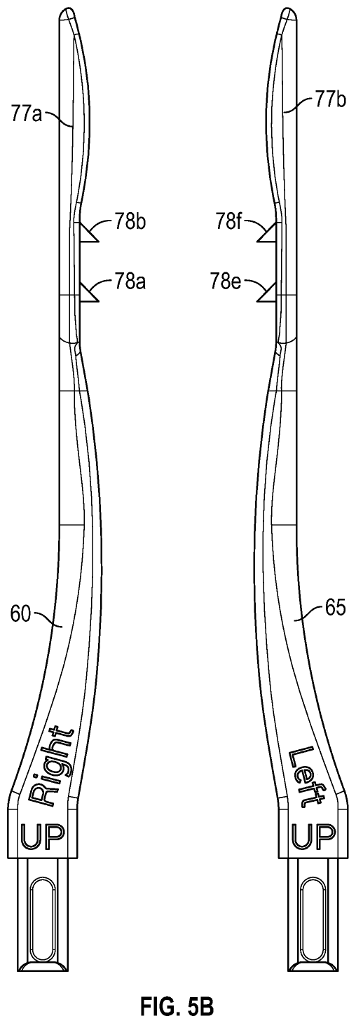

Turning to FIGS. 5A-5B, in some embodiments, the first and second jaw members 60, 65 may both include one or more tines 78a-h designed and/or configured to interact with each other to clamp bone tissue therebetween. As further depicted in FIGS. 5A-5B, in some embodiment, the first and second jaw members 60, 65 may both include a convex flange 77a, 77b extending distally beyond the tines 78a-h.

In some embodiments, as shown in FIGS. 1A and 7, the convex flange 77 may be longitudinally aligned with the tines 78a-d and/or the distal end part 33, 43 of one of the side members 30, 40. In some embodiments, as shown in FIGS. 5A-5B, one or more of the convex flanges 77a, 77b may be vertically aligned and/or not longitudinally aligned with the tines 78a-d and/or the distal end part 33, 43 of one of the side members 30, 40.

In some embodiments, as shown in FIGS. 1A and 7, the first and second jaw members 60, 65 may be of different configurations. In some embodiments, as shown in FIGS. 5A-5B, the first and second jaw members may be of the same design and/or configuration.

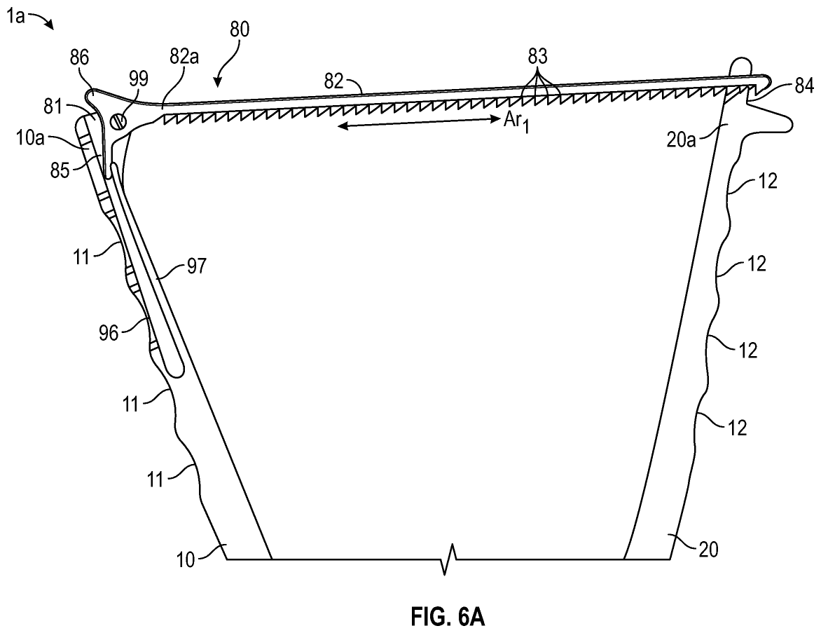

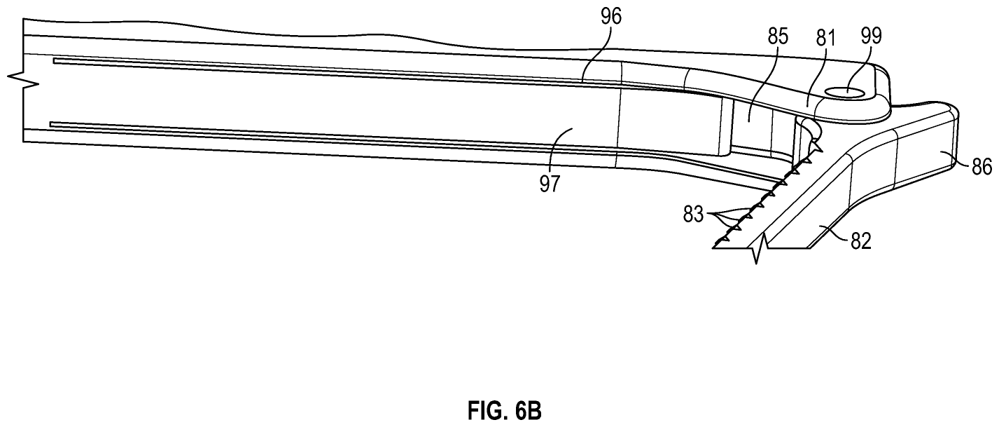

Turning to FIGS. 6A and 6B, a proximal end portion 1a of the surgical instrument 1 is shown in an open or receiving position and including a pivoting ratchet assembly 80 mounted to a collar 81 of the first arm 10 via a pin 99. The ratchet assembly 80 may include a straight locking arm 82 having a plurality of teeth 83 on at least one surface thereof. The teeth 83 configured to engage a complimentary tooth (or teeth) and/or pawl 84 positioned on and/or extending from a proximal end portion 20a of the second arm 20 to lock the first and second arms 10, 20 in a fixed position and/or lock the first and second jaw members 60, 65 relative to one another.

One end portion 82a of the locking arm 82 is pivotably coupled to the collar 81 and includes at least a first rib 85 and optionally a second rib 86 extending therefrom. In instances including a first and second rib 85, 86, the ribs extend in different directions from the end portion 82a of the locking arm 82 and are spaced from each other around an outer perimeter of the end portion 82a.

As shown in FIGS. 6A and 6B, the proximal end portion 10a of the first arm 10 includes a first passage 96 defined therein behind and/or surrounding a deflectable wall 97 defined on an inner surface of the first arm 10. The first rib 85 extends away from the end portion 82a into the first passage 96 and the optional second rib 86 may extend away from both the end portion 82a and the first arm 10, as well as the first rib 85. In some embodiments, the first rib may be longer and thinner than the second rib. In some embodiments, the first and second ribs are of the same shape and dimensions.

The first rib 85 is configured to interact with the deflectable wall 97 from behind the wall 97 and within the first passage 96. When an initial or minor force is applied outwardly (see arrow AR₁) on either of the first and/or second arms 10, 20 while in a locked position, the pinned end portion 82a will pivot causing the first rib 85 to rotate inwardly pushing into the backside of the deflectable wall 97 causing the wall 97 to deflect slightly inwardly while preventing the first rib from rotating further inwardly. This interaction between the rib 85 and the wall 97 prevents the locking mechanism 80 from simply unlocking when exposed to minor forces that may be associated with the handling of the instrument.

In some embodiments, the shape of the teeth and/or pawl 83 and 84 are such that when the first and second arms 10 and 20 are pressed inwardly towards each other, the teeth 83 can slide against the teeth or pawl 84 until a locked position is reached that maintains the graft between the jaw members. Once in a locked position, the interaction between the rib 85 and the wall 97 pushes and/or forces one side of one or more teeth 83 against one side of one or more teeth 84 preventing the locking mechanism 80 from simply unlocking due to the outward forces applied on the jaw by the graft which is in compression. For example, as depicted in FIG. 6A, each tooth of teeth 83, 84 may be defined by a longitudinal edge and a diagonal edge, the longitudinal edge extending generally perpendicular to arm 82. In a locked position, the interaction between the rib 85 and the wall 97 pushes and/or forces the longitudinal edge of tooth 83 against the longitudinal edge of tooth 84 preventing the locking mechanism 80 from simply unlocking due to outward forces applied on the jaw by the graft which may be under compression.

In some embodiments, the second rib 86 is configured to be a stop. For example, the second rib 86 may be closer to the first rib 85 than the locking arm 82 such that inward rotation of the first rib 85 will cause the second rib 86 to rotate until butting up against a proximal end portion 10a of the first arm 10 preventing or stopping any further rotation of either rib 85, 86.

In some embodiments, the second rib 86 may also be designed to ensure the user avoids pivoting the locking arm 82 and/or locking mechanism 80 too far when opening the ratcheting to release the graft between the jaw members.

In some embodiments, the second rib 86 is configured to be a release. For example, the second rib 86 may be closer to the locking arm 82 than the first rib 85 such that rotation of the second rib 86 will cause the locking arm 82 to pivot away from the pawl 84 unlocking or releasing the locking arm 82 from the second arm 20.

As further depicted in FIG. 6A (as well as FIGS. 1A-1C) each of the first and second proximal end portions 10a, 20a of the first and second arms 10, 20 may include one or more spaced grooves 11, 12 defined therein. The spaced grooves 11, 12, are configured to receive one or more of a user's fingers when handling the instrument 1.

In FIG. 7, in some embodiments, a surgical instrument 1, such as surgical forceps, may include first and second arms 10, 20 pivotably coupled to each other via a rotational joint 5. First and second side members 30, 40 are positioned on opposite sides of the rotational joint 5. A proximal portion of the first side member 30 pivotably connects a distal end portion 20b of the second arm 20 to the first arm 10 proximal the rotational joint 5. A proximal portion of the second side member 40 pivotably connects a distal end portion 10b of the first arm 10 to the second arm 20 proximal the rotational joint 5. A v-shaped linkage pivotably connects the first arm 10 to the second arm 20 proximal to the rotational joint 5. A first jaw member 60 is coupled to a distal portion 30b of the first side member 30 and a second jaw member 65 is coupled to a distal portion 40b of the second side member 40.

The surgical instrument 1 of FIG. 7 alternatively includes a guide assembly 70 made of only a single guide wall 71 including one or more bores 72, 73 extending completely therethrough. The single guide wall 71 is free of any gap.

FIG. 7 (and FIGS. 5A-5B) also illustrates that in some embodiments, the first and second jaw members 60, 65 may be configured to be interchangeable and/or removable, as opposed to being affixed to the instrument 1. By being interchangeable and/or removable, the same instrument 1 can be used for a surgical procedure on both a left and right joint simply by switching the first and second jaw members from one side to the other, as needed.

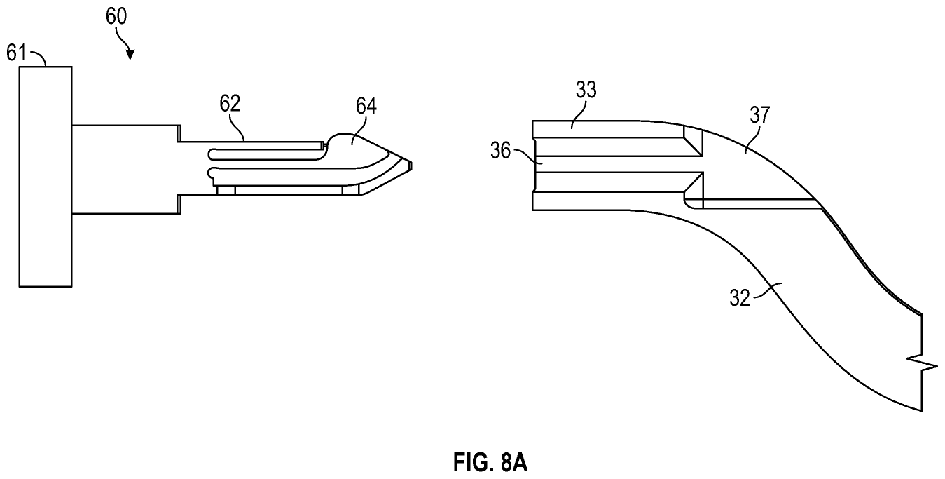

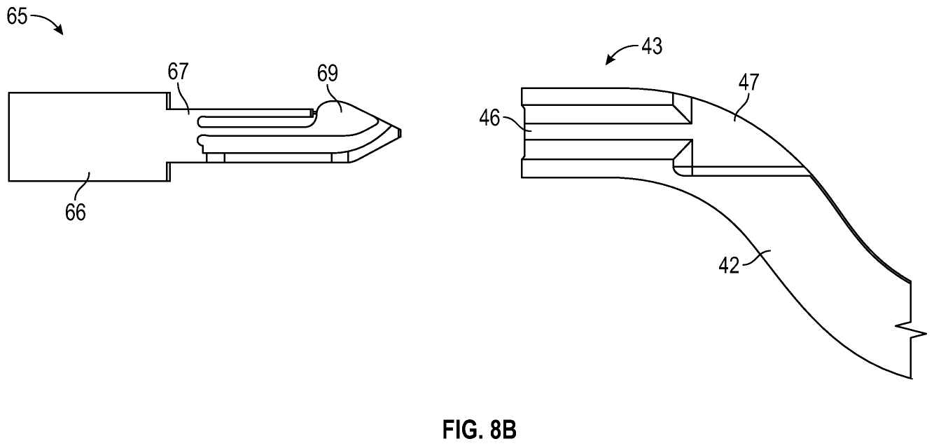

Turning to FIGS. 8A and 8B, in some embodiments, the first and second jaw members 60, 65 and the first and second distal portions 33, 43 of the instrument 1 are shown schematically in cross-section prior to being coupled. The first and second distal portions 33, 43 each include a member channel 36, 46 defined therethrough leading to a locking aperture 37, 47. Each member channel 36, 46 being configured to receive and maintain the locking post 62, 67 of the jaw member 60, 65 therein. Each locking aperture 37, 47 configured to receive and maintain a deflectable locking member 64, 69 therein locking the jaw member 60, 65 into place.

As shown in FIGS. 8A and 8B, each jaw member 60, 65 may include a jaw body 61, 66 extending longitudinally between a locking post 62, 67 on proximal end portion thereof and a shaped distal end portion 63, 68. Each jaw body 61, 66 defines a width greater than the respective locking post 62, 67. Each locking post 62, 67 is configured to fit and/or slide within the respective member channel 36, 46. The width of the each jaw body 61, 66 is configured to match the width of the respective planar distal end portion 33, 43.

As further shown in FIGS. 8A and 8B, each locking post 62, 67 includes one or more deflectable locking members 64, 69 configured to deflect inwardly to pass through the member channel 36, 46 and deflect outwardly when free of the member channel 36, 46 and particularly within the locking aperture 37, 47.

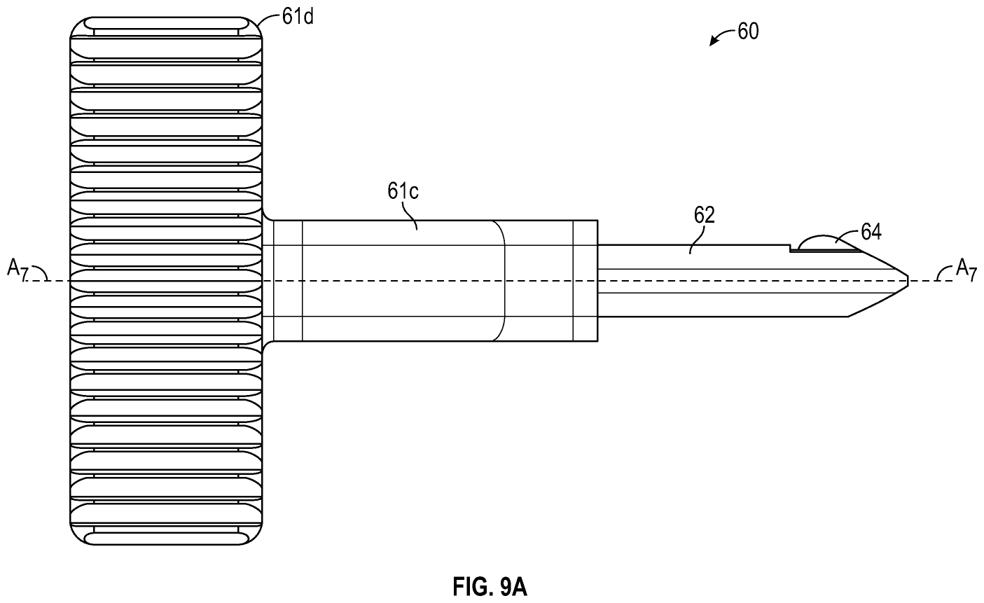

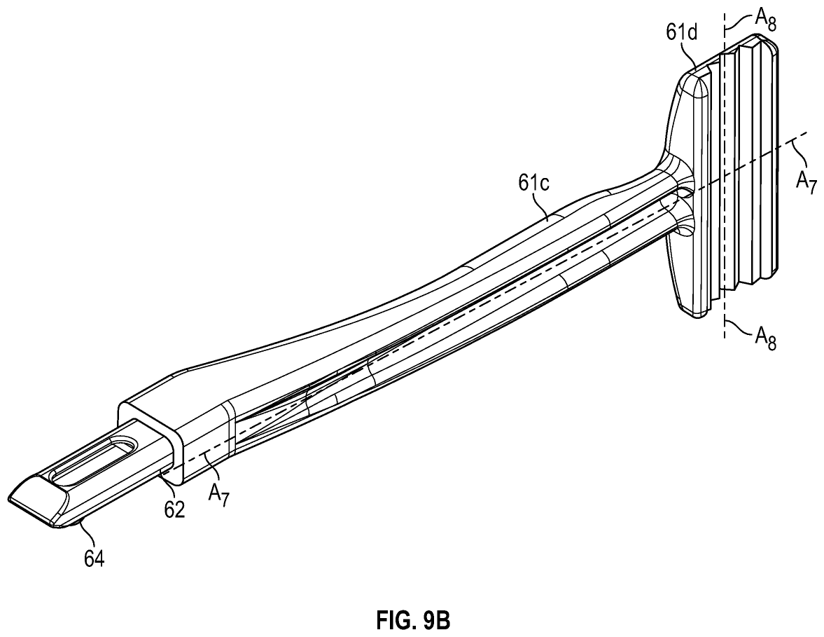

In some embodiments, as shown in FIGS. 9A and 9B, the first jaw member 60 may include a first jaw body 61 having a first part 61c defining a longitudinal axis A₇ (coplanar with the longitudinal axis A₃ of the second distal portion 33 of the first side member 30), and a second part 61d defining a longitudinal axis A₈ generally perpendicular to the longitudinal axis A₇ of the first part (or the second distal portion 33 of the first side member 30). In some embodiments, as particularly depicted in FIG. 9A, the treads 79 may extend generally parallel to the longitudinal axis A₇ of the first part 61c. In some embodiments, as particularly depicted in FIG. 9B, the treads 79 may extend generally parallel to the longitudinal axis A₈ of the second part 61d.

The instruments described herein may include any combination of the first and second jaw members described herein. Each of the jaw members may be configured to be affixed, removable, and/or interchangeable as needed.

In FIG. 7, the surgical instrument 1 is shown in an open or receiving position. In the open or receiving position, the first and second proximal end portions 10a, 20a of the surgical instrument 1 of FIG. 7 are spaced farthest apart from each other and so are the first and second jaw members 60, 65.

In FIG. 10, the surgical instrument 1 is shown locked in a closed or clamping position. In the closed or clamping position, the first and second proximal end portions 10a, 20a are positioned closest to each other and so are the first and second jaw members 60, 65. The surgical instruments described herein are configured to transition between the open and closed positions, as needed.

Although the first and second arms 10, 20 are on a first plane different than a second plane of the first and second jaw members 60, 65, lateral linear movement of the arms 10, 20 along the first plane translates into lateral linear movement of the jaw members 60, 65 along the second plane. This may be possible because the side members 30, 40 remain generally parallel to each other during the lateral linear movement of the arms 10, 20 and/or jaw members 60, 65. In addition, the movement of the arms 10, 20 and jaw members 60, 65 occurs around the guide assembly 70 which remains substantially stationary relative to the arms 10, 20, and jaw members 60, 65, as well as generally centrally positioned therebetween.

FIGS. 11-15 illustrate a plurality of steps of a surgical procedure such as a Latarjet procedure carried out by using a surgical instrument 1, and particularly a clamping device such as a surgical forceps, as described in at least one embodiment herein. As explained above, one benefit of the novel features of the instrument 1 is that of carrying out a method which is remarkable in that it consists of positioning by means of a surgical instrument 1 a bone graft G on the scapula S and drilling said bone graft G at the same time as the scapula S with the aid of the guide assembly 70 including bores 72, 73 borne by the same surgical instrument 1. FIGS. 11-13 illustrate such a method with beforehand the removal of the coracoid in order to have a bone graft G to be attached to the scapula.

As illustrated in FIG. 11, a pair of retractors 59 maintain access to the coracoid process 56 for cutting with a bone-cutting device 57, such as a sagittal saw equipped with a 90 degree blade. A periosteal elevator 58 may be used to expose the knee of the coracoid process 56. A coracoacromial ligament stump CL remains attached to the coracoid process 56 (and/or the bone graft G).

The surgical instruments 1 described herein may be used before, during, and/or after the cutting of the coracoid process 56 to clamp or grasp the coracoid graft G in a generally fixed position. It is envisioned that the instrument 1 will approach the coracoid process 56 straight on with a front face 56a of the coracoid 56 positioned between with the first and second jaw members of the instrument. In this configuration, the bone graft G may not need to be further manipulated and/or rotated prior to reattachment to the scapula S.

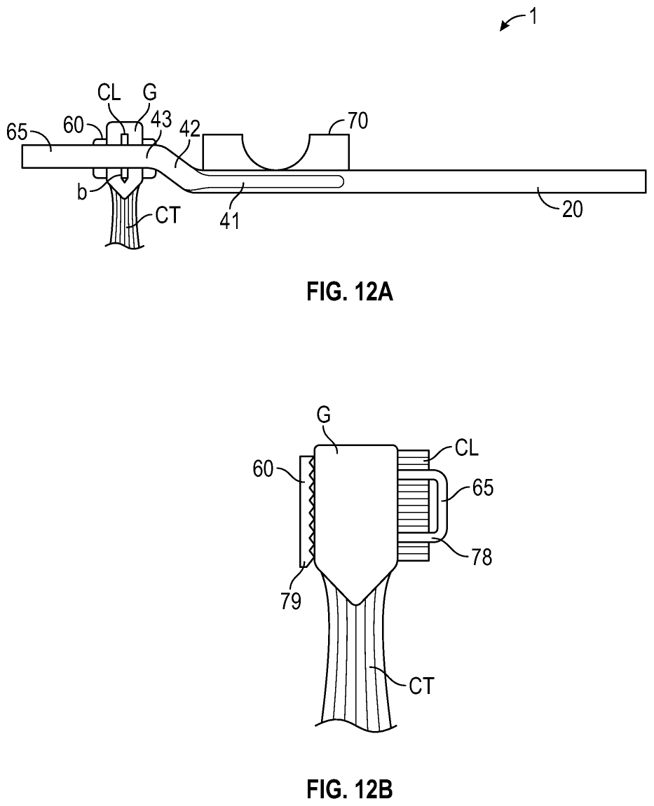

FIGS. 12A and 12B illustrate the position of coracoid or bone graft G within the jaw members 60, 65 of the surgical instrument 1 described herein. The bone graft G may include the coracoacromial ligament stump CL and remain attached to the conjoined tendon CT. As noted hereinabove, the combination of the tines 78 and the concave surface of the second jaw member 65 creates extra and/or sufficient space therebetween for the coracoacromial ligament stump CL to be positioned without damage, while the graft G is secured between the treads 79 on the first jaw member 60 and the tines 78 on the second jaw member 65. In this position, a majority of the bone graft G is accessibly for resurfacing, if needed.

Alternatively, in some instances, the bone graft G may also be rotated and/or repositioned within the instrument 1 to be resurfaced. To do this, the bone graft G is turned in the forceps to have more access to the surface to be freshened.

As illustrated by FIGS. 13A-13C, to secure the bone graft G to the front face of the scapula S, the graft G may be locked between the jaw members 60, 65 with the convex flange 77 positioned bearing against the generally concave glenoid joint surface GL of the scapula S. When the convex flange 77 is properly seated in the glenoid joint GL, the proper placement of the bone graft G on the front face FF of the scapula S is likely, if not a certainty.

As shown in FIG. 13B, when the instrument 1 positions the bone graft G on the front face FF of the scapula S, the guide assembly 70, and particularly the bores 72, 73, are centrally aligned and/or centered on the front face of the bone graft G. A drill bit (not shown) can be passed through either or both of the bores 72, 73 to drill holes through the graft G and also the scapula S.

After producing the drill holes, the instrument may be removed while the drill bits are retained in the drill holes. The drill bits may then be successively removed and replaced with any suitable fastener, such as a compression screw.

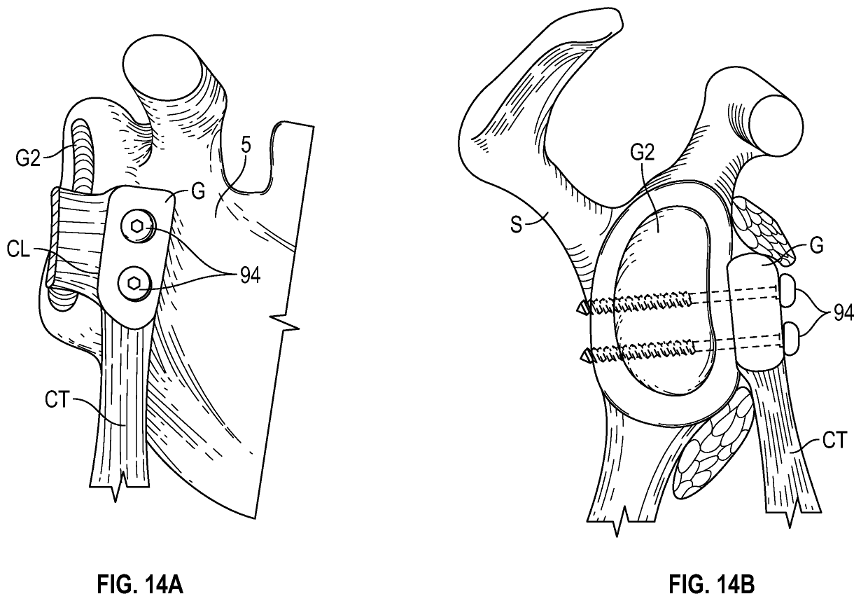

As shown in FIGS. 14A and 14B, two fasteners 94, such as compression screws, affix the bone graft G to the scapula S with the conjoined tendon in tact and the coracoacromial ligament extending therefrom and ready to be reattached.

As shown in FIG. 13C, the surgical instrument 1 is shown in an upright position configured to be used on a right shoulder of a patient and includes the second jaw member 65 with the flange 77 attached to the second side member 40 and the first jaw member 60 (without the flange) attached to the first side member 30. In this configuration, with respect to the right scapula (and/or shoulder), the second jaw member 65 with the flange 77 is positioned on a lateral side of the right scapula (and/or shoulder) and the first jaw member 60 (without the flange 77) is positioned on a medial side of the right scapula (and/or shoulder). This configuration allows the flange 77 to interact with the glenoid joint GL on the lateral side of the scapula S while the other jaw member (without the flange) avoids other bony parts of the scapula S on the medial side.

In some embodiments, the surgical instrument may be configured to be used on a left shoulder. In such embodiments, the surgical instrument, in an upright position, may include the second jaw member with the flange attached to the first side member and the first jaw member (without the flange) attached to the second side member 40. In this configuration, with respect to the left scapula (and/or shoulder), the second jaw member with the flange is positioned on a lateral side of the left scapula (and/or shoulder) and the first jaw member (without the flange) is positioned on a medial side of the left scapula (and/or shoulder). This configuration allows the flange to interact with the glenoid joint on the lateral side of the left scapula while the other jaw member (without the flange) avoids other bony parts of the left scapula on the medial side.

In some embodiments, since the jaw members may be removable, replaceable, and/or interchangeable, the same general instrument may be used for either a left or right side of the patient simply by switching sides of the first and second jaw members, as needed.

In addition, rather than changing the location of the jaw members, in some embodiments, the surgical instrument 1 of FIGS. 13A-13C may also be configured to be used in a left shoulder procedure. However, instead of being used in an upright position, i.e., as shown with the guide assembly sitting on top of the rotational joint, the surgical instrument 1 may be used in an inverted or upside-down configuration, i.e., as shown in FIG. 15 wherein the rotational joint sits on top of the guide assembly 70. Since the movement of the jaw members is linear and/or the guide assembly is affixed in a position centrally aligned between the jaw members, inverting the instrument 1 does not prevent the instrument 1 from being equally effective in an inverted configuration (FIG. 15) as when in the upright position (FIG. 13A-13C). Also, since the coracoid is shortened to obtain the bone graft G, the inverted instrument 1 should be clear of the other bony parts of the scapula S.

In some embodiments, the surgical instruments described herein may be configured to be used in an upright configuration and an inverted configuration.

II. Surgical Clamp Pistol Design

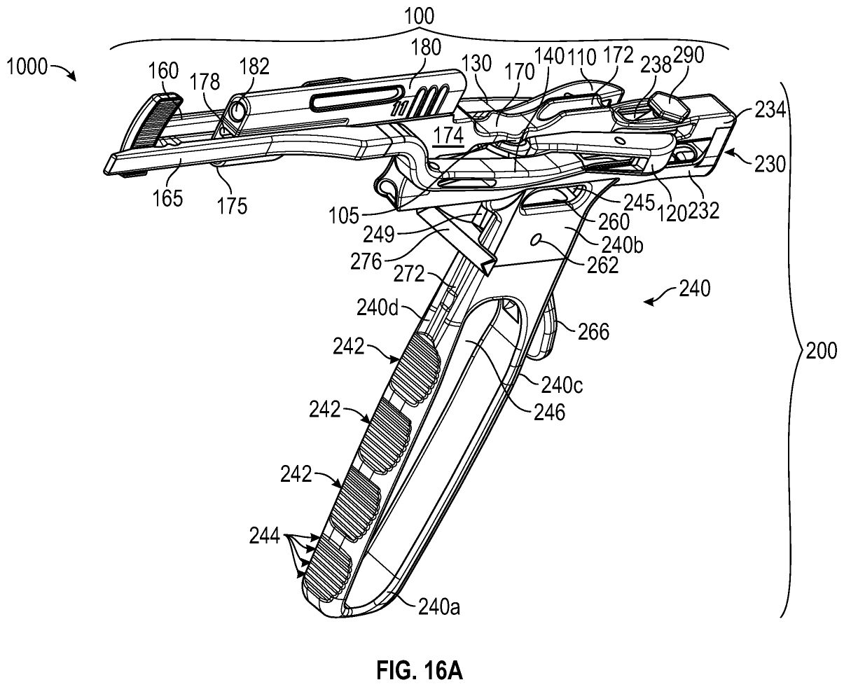

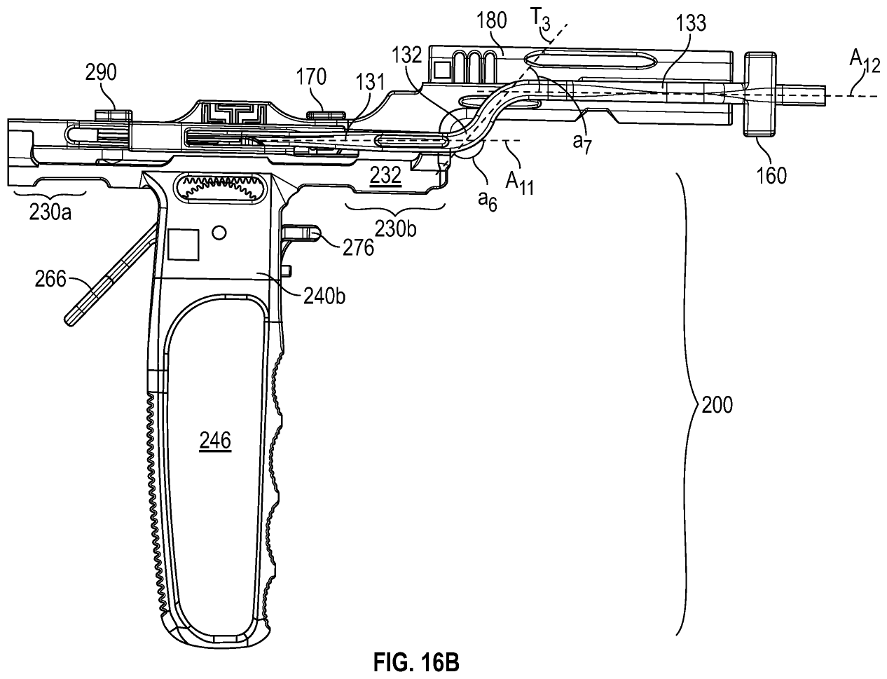

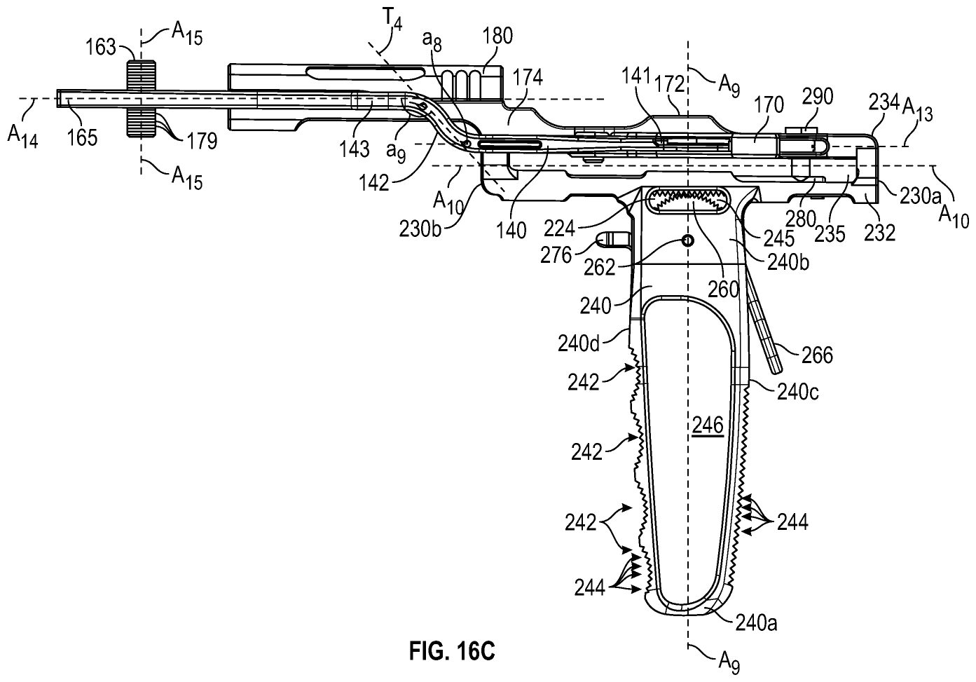

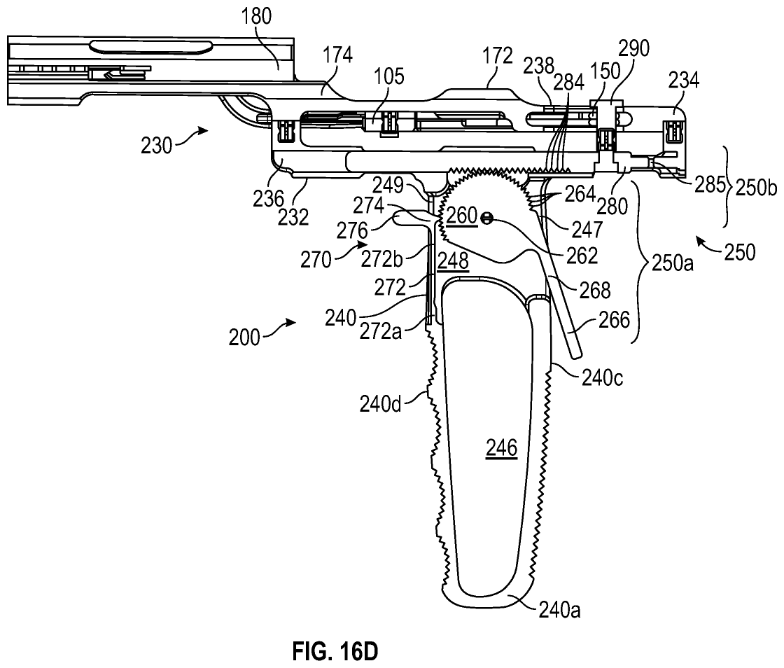

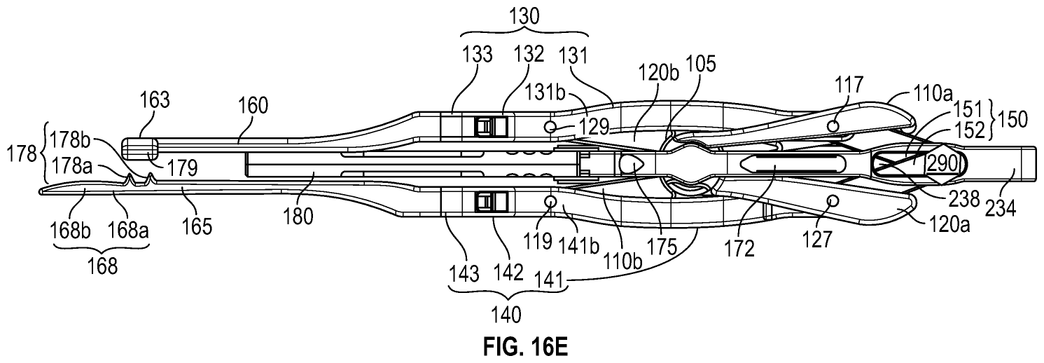

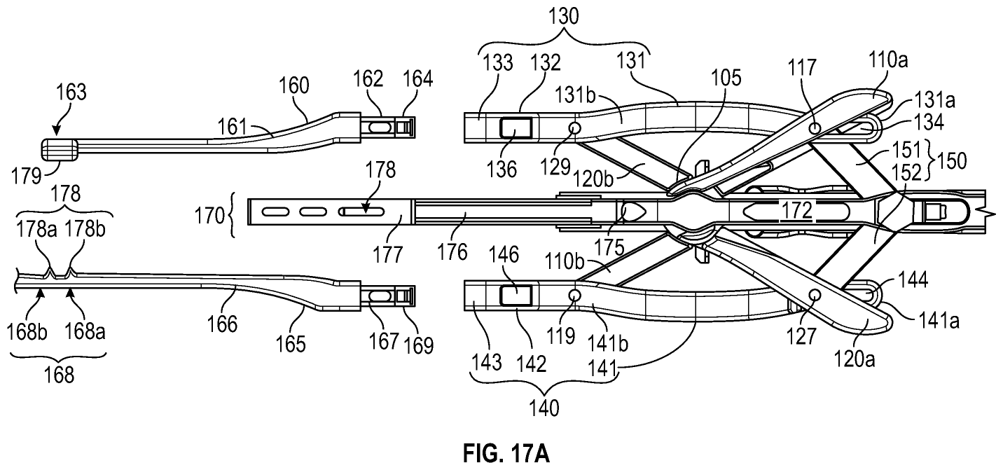

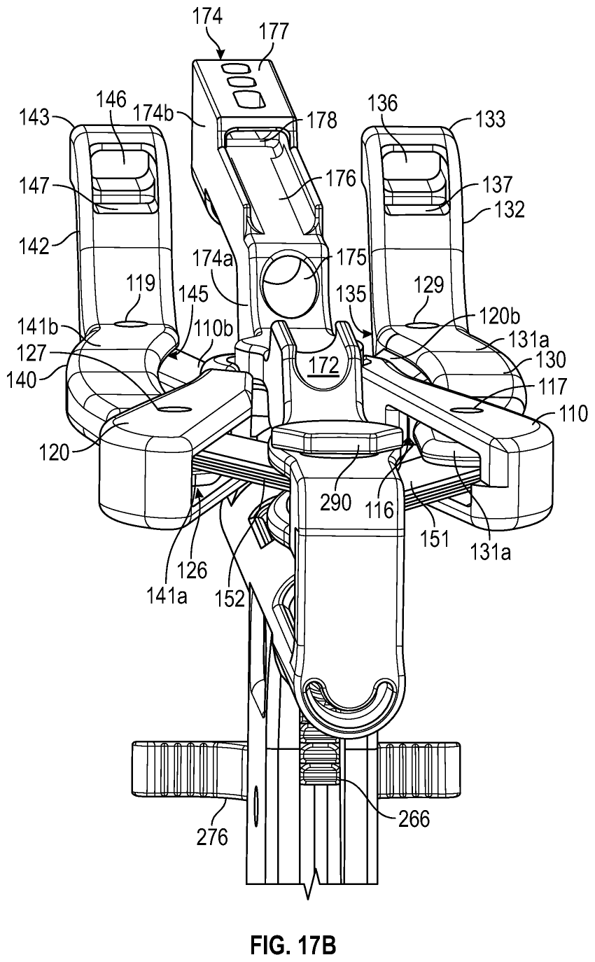

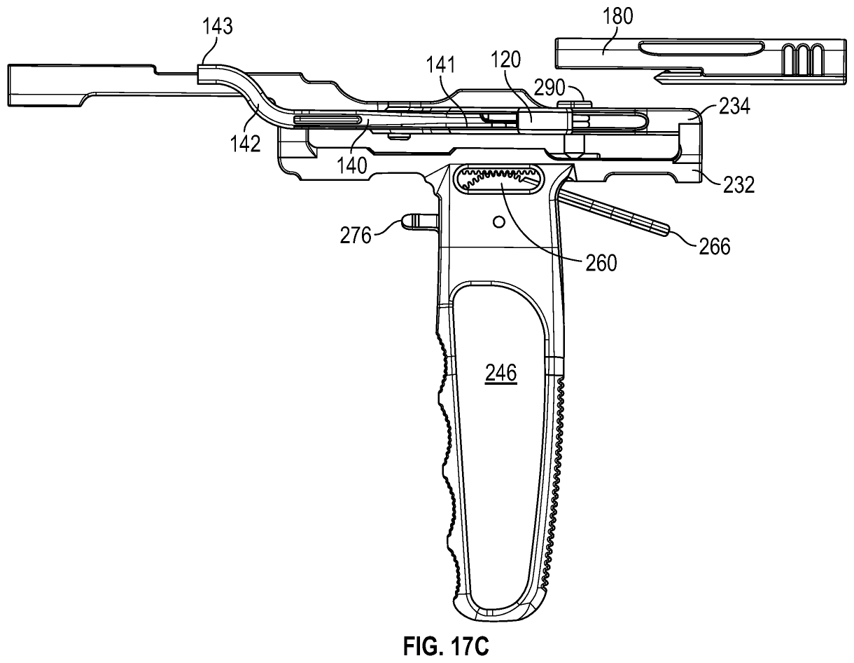

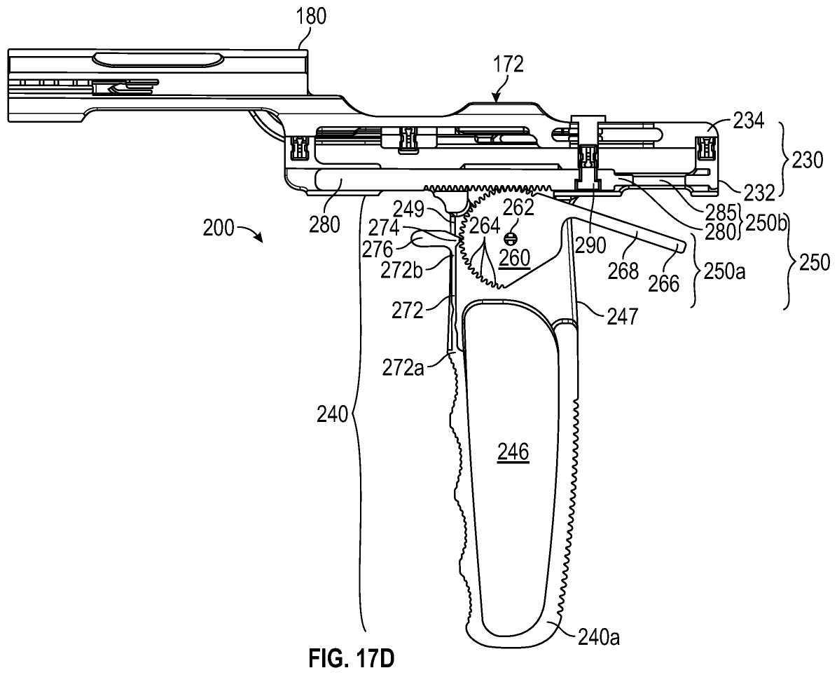

With reference to FIGS. 16A-17D, illustrated is an assembled and operable surgical instrument 1000, and particularly a surgical clamp in a surgical pistol design 1000, according to at least one embodiment of the present disclosure. The surgical instrument 1000 is configured to transition between a closed or clamping configuration (as depicted in FIGS. 16A-16E) and an open or non-clamping configuration (as depicted in FIGS. 17A-17D). As shown, in some embodiments, the surgical instrument 1000 may include a handle portion 200 operably coupled to a clamp assembly 100 via gear assembly 250. The instrument 1000 may further include a guide assembly 170.

A. Handle Portion

As illustrated in at least FIG. 16A, the surgical instruments 1000 in a pistol design as described herein may include a handle portion 200 that is ergonomic in design. In some embodiments, the surgical instrument 1000 may include a pistol-grip type handle portion 200.

The handle portion 200 includes a support frame 230 positioned on top of a handle body 240. The handle body 240 extends along an axis A₉, transverse a longitudinal axis A₁₀ of the support frame 230, and between a first end portion 240a and a second end portion 240b defining a length of the handle body 240. The first end portion 240a of the body 240 is free of the support frame 230, the clamp assembly 100, and/or the gear assembly 250. The second end portion 240b, on the other hand, is connected to the support frame 230 and houses at least a portion of the gear assembly 250. The handle body 240 also extends transversely between a proximal handle face 240c and an opposite distal handle face 240d defining a width of the handle body 240 therebetween.

In some embodiments, the handle portion 200 and/or handle body 240 is configured to be manually-operated. In some embodiments, the handle portion 200 and/or handle body 240 is free of a power source, a motor, and/or electrical wiring.

The handle body 240 (and/or at least the first end portion 240a of the handle body 240) is configured to held by a hand of a user, such as a surgeon or other medical personnel. The distal handle face 240d may include one or more finger grooves 242 defined therein. The grooves 242 are configured to receive one or more of a user's fingers when handling the instrument 1000. The finger grooves 242 being spaced along the distal handle face 240d. Each of the grooves 242 may further include a texture 244, such as a series of smaller ridges, designed to increase the surface area of the grooves 242 for a more secure grip and better handling of the instrument 1000. In some embodiments, the texture 244 may also be positioned along the proximal handle face 240c of the first end portion 240a. However, the proximal handle face 240c is free of grooves 242 in order to properly receive a portion of the user's palm, as opposed to the user's fingers, against thereof when properly grasped.

The first end portion 240a of the handle body 240 may further include a handle aperture 246 defined therethrough. However, in some embodiments, the first end portion of the handle body may be solid and/or free of a handle aperture.

The handle body 240, and particularly the second end portion 40b of the handle body 240, may define a handle cavity 248 therein. In some embodiments, the handle cavity 248 may be located on top of the handle aperture 246. The handle cavity 248 is designed to contain at least a first portion 250a of the gear assembly 250 therein (FIGS. 16D and 17D). The first portion 250a of the gear assembly 250 includes at least a first gear 260, a trigger 266, and a locking member 270.

The handle body 240, and particularly the second end portion 240b of the handle body 240, may further include at least one handle window 245 defined therethrough to provide visibility of at least a portion of the first gear 260 from outside the cavity 248. In some embodiments, the handle window 245 is generally oblong in shape and provides visibility of the interaction between the first gear 260 and the second gear 280, and particularly the interaction between the first and second set of teeth of the first and second gears 260, 280. It is envisioned that one or more of the first and/or second gears 260, 280 may further include a visual indicator designed to be seen through the handle window 245 indicating the current configuration of the instrument, i.e., clamping or non-clamping, closed or open, etc.

The handle body 240, and particularly the second end portion 240b of the handle body 240, may further include a first handle slot 247 defined through a portion of the proximal face 240c. The first handle slot 247 being configured to allow the trigger 266 (and/or trigger arm 268) to extend therethrough in a proximal direction from the first gear 260 located inside the handle cavity 248 to outside the handle body 240.

The handle body 240, and particularly the second end portion 240b of the handle body 240, may further include a second handle slot 249 defined through a portion of the distal face 240d. The second handle slot 249 being configured to allow the locking member 270 to extend therein and therethrough as needed to lock and/or unlock the rotation of the first gear 260.

The second end portion 240b of the handle body 240 may also be affixed to the support frame 230. The support frame 230 and the handle body 240, in the pistol design, are in fixed relation to one another and/or non-movable relative to each other.

The support frame 230 extends along a longitudinal axis A₁₀ between a proximal end portion 230a and a distal end portion 230b defining a length of the support frame 230. The support frame 230 includes at least a base 232 and a cover 234 connected to each other at least along proximal and/or distal end portions 230a, 230b of the support frame 230. In some embodiments, the base 232 and the cover 234 may be separated by a frame gap 235 therebetween along a portion of the frame 230 length.

In some embodiments, the handle portion 220 may be a monolithic structure wherein the cover 234, the base 232 and the handle body 230 are a one-piece design. In some embodiments, the cover 234 is configured to be affixed to the base 232, such as via pins, screws, bolts, adhesives, snap-fitting, or the like.

In some embodiments, the support frame 230 of the pistol design may include a guide assembly 170 on a distal end portion 230b thereof. In some embodiments, the guide assembly 170 (not including the removable guide insert 180) may be monolithic with the support frame 230. In some embodiments, the guide assembly 170 (not including the removable guide insert 180) may be configured to be affixed to the cover 234, such as via pins, screws, bolts, adhesives, snap-fitting, or the like.

In the pistol design, the axis A₉ of the handle body 240 (and/or handle portion 200) and the longitudinal axis A₁₀ of the support frame 230 cross to form a fifth angle a₅ ranging from about 25-135°. In some embodiments, the fifth angle a₅ may range from about 35-105°. In some embodiments, the fifth angle a₅ may range from about 45-95°. In some embodiments, the fifth angle a₅ may be about 90°. In some embodiments, the handle body 240 and the support frame 230 generally form a T-shaped handle portion 200.

In the pistol design, the support frame 230, and particularly the cover 234, is configured to secure at least a portion of the clamp assembly 100 thereto as provided in more detail hereinbelow.

The support frame 230, and particularly the base 232, is configured to house a second portion 50b of the gear assembly 50 as provided in more detail hereinbelow. In some embodiments, the base 32 includes a first gear channel 36 defined therein, the first gear channel 36 configured to receive at least a second gear 66 therein.

B. Gear Assembly

As provided hereinabove and best shown in FIGS. 16D and 17D, the gear assembly 250 is located within the handle portion 200. The gear assembly includes a first lower gear portion 250a generally located in the handle body 240 and a second upper portion 250b generally located in the support frame 230. The first and second portions 250a, 250b of the gear assembly 250 are designed to work together to cause the clamp assembly 100 (and/or the jaw members 160, 165) to transition between a closed or clamping configuration (FIGS. 16A-16D) and an open or non-clamping configuration (FIGS. 17A-17D).

The first portion 250a of the gear assembly 250 may include at least a first gear 260, a trigger 266, and a locking member 270. The first gear 260 is configured to rotate about pivot point 262 and includes gear teeth 264 along an outer perimeter (or circumference) thereof. The gear teeth 264 may partially or completely wrap around the outer perimeter (or circumference) of the first gear 260 (not including the trigger arm 268).

The first gear 260 is located within the handle cavity 248 (and/or the second end portion 240b of the handle body 240) and is affixed to the trigger 266 by a trigger arm 268. The trigger arm 268 extends from the first gear 260 inside the handle cavity 248, through the proximal handle slot 247, to the trigger 266 positioned outside the handle cavity 248. The trigger arm 268 is designed to pivot and/or rotate within the proximal handle slot 247 such that the trigger 266 can be moved from near the base 232 of the support frame 230 (FIG. 17D) to near the user's hand when ergonomically holding the handle body 240 (FIG. 16D). In such a design, the user may apply pressure, particularly with their thumb, to the trigger 266 to cause, via the trigger arm 268, rotation of the first gear 260 about the pivot point 262.

The first portion 250a of the gear assembly 250 further includes a locking member 270 including at least a locking arm 272, a locking pawl 274, and a tab member 276. The locking arm 272 extends between a first fixed end portion 272a secured to and/or extending from the handle body 240 and a second free end portion 272b including the tab member 276 and the locking pawl 274 on opposite sides thereof. The locking arm 272 may be positioned within and extend along a length of the distal handle slot 249 with the tab member 276 extending out of the distal handle slot 249 while the locking pawl 274 remains generally in the handle cavity 248 to interact with the first gear 260, and particularly a portion of the first set of gear teeth 264 of the first gear 260.

The locking pawl 274 is shaped and/or configured to interact with one or more of the gear teeth 264 of the first gear 260 to prevent rotation of the first gear 260. The tab member 276 is designed to remain outside the handle body 240 and accessible to a user. The locking arm 272 is designed to pivot away from the first gear 260 when pressure is applied to the tab member 276. For example, the user may use one or more of their fingers, e.g., pointer finger and/or middle finger, to press on the tab member 276 releasing the locking pawl 274 from the gear teeth 264 thereby allowing the rotation of the first gear 260.

The second upper portion 250b of the gear assembly 250 includes at least a second gear 280, a biasing member 285, and a clamp pin 290 connecting the second gear 280 to the clamp assembly 100. The clamp pin 290 extends from the second gear 280 to a pin slit 238 defined in the cover 234, with a proximal part of the v-shaped linkage 150 positioned therebetween.

The second gear 280 is configured to slide side to side, i.e., proximally or distally, relative to the first gear 260 within the gear channel 236 of the support frame 230. A second set of gear teeth 284 of the second gear 280 are located along at least a portion of an outer surface of the second gear 280. The gear teeth 284 being configured to interact with the first set of gear teeth 264 of the first gear 260 such that when the first gear 260 is rotated in either direction, i.e., clockwise or counter-clockwise, the second set of gear teeth 284 will force the second gear 280 to slide proximally or distally relative to the first gear 260 and/or within the gear channel 236. In some embodiments, the first and second gears 260, 280 are a rack and pinion type gear combination. In some embodiments, the first gear includes an outer perimeter defining a circular, oval, or polygonal shape, while the second gear defines a generally linear shape.

The clamping pin 290 is also configured to slide side-to-side within the pin slit 238 (and/or the support frame 230). Since the clamping pin 290 extends from a proximal end portion of the second gear 280 and passes through a proximal part of the v-shaped linkage 150, the sliding of the second gear 280 causes the clamping pin 290 to also slide side-to-side, which further causes the proximal part of the v-shaped linkage 150 to move proximally or distally relative to the other components of the clamp assembly (i.e., the first and second arms 110, 120, the first and second side members 130, 140, and/or the first and second jaw members 160, 165). The proximal and/or distal movement of the v-shaped linkage 150 causes the other components of the clamp assembly to pivot forcing the jaw members to open or close and/or transition the surgical instrument between an non-clamping configuration and a clamping configuration.

As best shown in at least FIGS. 16D and 17D, in some embodiments, the biasing member 285 may be positioned in a proximal end portion of the base 232 and/or proximal the second gear 280. In some embodiments, the biasing member 285, such as a compression spring, may be configured to force the second gear 280 in a distal direction within the channel 236. In such embodiments, the first gear 260 may be configured to rotate in a clockwise manner moving the second gear 280 in a proximal direction against the biasing member 285, while the locking member 270 prevents the first gear 260 from rotating counterclockwise under the force applied by the biasing member 285 in the distal direction against the second gear 280. This design provides a ratcheting configured to allow incremental changes, without risk of slip, while transitioning between the non-clamping and clamping configurations. For example, a user may apply downward pressure on the trigger 266, causing clockwise rotation of the first gear 260, while transitioning the instrument from a non-clamping configuration to a clamping configuration. Alternatively, a user may apply pressure to the tab member 276 causing the locking pawl 274 to disengage the gear teeth 264 of the first gear 260, allowing counter-clockwise rotation of the first gear 260, while transitioning the instrument from a clamping configuration to a non-clamping configuration. The counter-clockwise rotation of the first gear 260 being driven by the force applied thereto, via the second gear 280, by the biasing member 285. This configuration also provides the jaw members 160, 165 of the clamp assembly 100 the force required to clamp and/or maintain an object therebetween without having to maintain pressure on the trigger.

In some alternative embodiments, the biasing member 285 may be positioned in a distal end portion of the base 232 and/or distal the second gear 280. In such embodiments, the biasing member 285, such as a tension spring, may be configured to apply pressure against the second gear 280 sliding in a proximal direction within the channel 236 and/or to force the second gear 280 to slide distally when the locking member 270 is released. This configuration may also provide the ratcheting described immediately hereinabove.

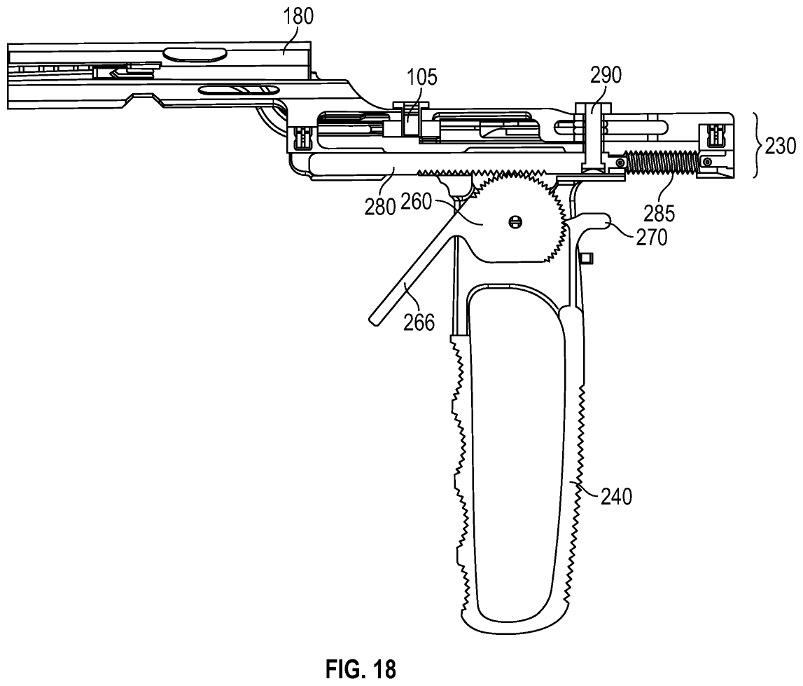

As further shown in FIG. 18, in some embodiments, the first gear 260, the trigger 266 and the locking member 270 may be reversed and/or located on opposite sides of the handle body 240. In such embodiments, pressing down on the trigger 266 may cause the first gear 260 to rotate counter clock-wise, which may cause the second gear 280 to slide distally within gear channel 236. As shown, the biasing member 285, such as a tension spring, may be located proximal the second gear 280 to apply pressure against the second gear 280 sliding in a distal direction within channel 236 and/or to force the second gear 280 in a proximal direction when the locking member 270 is released. This configuration may also provide the ratcheting described immediately hereinabove.

Alternatively, it is envisioned that the biasing member 285, such as a compression spring, may be located distal the second gear 280 to force the second gear 280 in a proximal direction when the locking member 270 is released.

C. Clamp Assembly

As can be seen from at least FIGS. 16A, 16E, 17A, and 17B, the surgical instruments 1000 in the pistol design or configuration as described herein include a clamp assembly 100 made up of a variety of interconnected clamp components which are directly or indirectly attached to the support frame 230. Some non-limiting examples of the various clamp components may include: a first and second arm 110, 120, a first and second side member 130, 140, a first and second link member 151, 152, a first and second jaw members 160, 165, and/or a guide assembly 170. The clamping pin 290 operably coupled to the first and second link members 151, 152 in a manner which may drive the various clamp components of the clamp assembly 100 to transition between the closed or clamping configuration (FIGS. 16A-16E) and the open or non-camping configuration (FIGS. 17A-17D).

Each of the components of the surgical apparatus 1000 may be manufactured from any suitable sterilizable material including, but not limited to, stainless steel, silver, titanium, nitinol, and the like. Any suitable polymeric material may also be used including, but not limited to, polyether ether ketones (PEEK), polycarbonates, polyphenylsulfones (Rader)) and the like.

As best shown in FIGS. 16A, 16E, 17A, and 17B, the clamp assembly 100 includes a first arm 110 and a second arm 120 pivotably coupled to each other. The first and second arms 110, 120 are configured to pivot and/or rotate around a rotational joint 105 for opening and/or closing of the instrument 100, and more particularly, the opening and/or closing of a first and second jaw member 160, 165. The first and second arms 110, 120 overlap and/or cross over each other at the rotational joint 105. In some embodiments, the rotational joint 105 may be centered along a length of each the first and second arms 110, 120.

In some embodiments, a first length between the first proximal pin 117 and the rotational joint 105 along the first arm 110 is equal to a second length between the first distal pin 119 and the rotational joint 105 along the first arm 110. In some embodiments, a third length between the second proximal pin 127 and the rotational joint 105 along the second arm 120 is equal to a fourth length between the second distal pin 129 and the rotational joint 105 along the second arm 120. In some embodiments, the first, second, third, and fourth arm lengths may be equal.

In some embodiments, a fifth length between the tip of the distal end portion 110b and the rotational joint 105 along the first arm 110 is less than a sixth length between the tip of the proximal end portion 110a and the rotational joint 105 along the first arm 110. In some embodiments, the fifth length is about 50 to 90% less than the sixth length. In some embodiments, the fifth length is about 65 to 75% less than the sixth length. In some embodiments, the fifth length is about 70% less than the sixth length.

In some embodiments, a seventh length between the tip of the distal end portion 120b and the rotational joint 105 along the second arm 120 is less than an eighth length between the tip of the proximal end portion 120a and the rotational joint 105 along the second arm 120. In some embodiments, the seventh length is about 50 to 90% less than the eighth length. In some embodiments, the seventh length is about 65 to 75% less than the eighth length. In some embodiments, the seventh length is about 70% less than the eighth length.

In addition to the first and second arms 110, 120, the clamp assemblies 100 described herein further include first and second side members 130, 140 and a v-shaped linkage 150 including a first and second link member 151, 152.

Each of the first and second side members 130, 140 includes: a proximal portion 131, 141 pivotably connected to both the first and second arms 110, 120; a distal portion 133, 143 configured to connect to a jaw member 160, 165; and an offset portion 132, 142 positioned therebetween.

In some embodiments, the first side member includes a central offset portion 132 between the proximal and distal end portions 131, 133 defining a first offset side member 130. In some embodiments, the second side member 140 includes a central offset portion 142 between the proximal and distal end portions 141, 143 defining a second offset side member 140.

In some embodiments, the proximal portion 131 of the first side member 130 extends between a first end part 131a and an opposite second end part 131b, the first end part 131a being pivotably coupled to a portion of the first arm 110 proximal the rotational joint 105, and the opposite second end part 131b being pivotably coupled to the distal end portion 120b of the second arm 120.

In some embodiments, the proximal portion 141 of the second side member 140 extends between a first end part 141a and an opposite second end part 141b, the first end part 141a being pivotably coupled to a portion of the second 120 proximal the rotational joint 105, and the opposite second end part 141b being pivotably coupled to the distal end portion 110b of the first arm 110.

As shown in FIGS. 17A and 17B, the first end part 131a (of the proximal portion 131 of the first side member 130) includes a first member opening 134 defined therethrough, wherein at least a some of the first end part 131a and/or the first member opening 134 is received within a first channel 116 defined through the first arm 110. The first channel 116 being positioned proximal to the rotational joint 105. A first proximal pin 117 passes perpendicularly through the first channel 116 of the first arm 110 and the first member opening 134 of the first side member 130 to pivotably couple the first side member 130 to the first arm 110. The first proximal pin 117 also passes through a distal end portion of the first link member 151 located within the first channel 116 beneath the first end part 131a.

As further shown in FIGS. 17A and 17B, the second end part 131b (of the proximal portion 131 of the first side member 130) includes a first slot 135 defined therein, wherein at least a portion of the second distal end portion 120b of the second arm 120 is received within the first slot 135. A second distal pin 129 passes perpendicularly through the first slot 135 of the first side member 130 to pivotably couple the second end part 131b of the first side member 130 to the second distal end portion 120b of the second arm 120.

As still further shown in at least FIGS. 17A and 17B, the first end part 141a (of the proximal portion 141 of the second side member 140) includes a second member opening 144 defined therethrough, wherein at least a some of the first end part 141a and/or the second member opening 144 is received within a second channel 126 defined through the second arm 120. The second channel 126 being positioned proximal to the rotational joint 105. A second proximal pin 127 passes perpendicularly through the second channel 126 of the second arm 120 and the second member opening 144 of the second side member 140 to pivotably couple the second side member 140 to the second arm 120. The second proximal pin 127 also passes through a distal end portion of the second link member 152 located within the second channel 126 over the first end part 141a.

The second end part 141b (of the proximal portion 141 of the second side member 140) includes a second slot 145 defined therein, wherein at least some of the first distal end portion 110b of the first arm 110 is received within the second slot 145. A first distal pin 119 passes perpendicularly through the second slot 145 of the second side member 140 to pivotably couple the second side member 140 to the first distal end portion 110b of the first arm 110.

As further shown at least in FIGS. 16A-16B, the first offset portion 132 (of the first side member 130) is positioned between a generally planar first proximal portion 131 and a generally planar first distal portion 133. The first proximal portion 131 of the side member defines a longitudinal axis A₁₁ and the first distal portion 133 defines a longitudinal axis A₁₂ offset from the longitudinal axis A₁₁ of the first proximal portion 131. The longitudinal axes A₁₁, A₁₂ being generally parallel to each other.

The first offset portion 132 defines a transverse axis T₃ extending at a sixth angle a₆ relative to longitudinal axis A₁₁ of the first proximal portion 131 and/or extending at a seventh angle a₇ relative to the longitudinal axis A₁₂ of the first distal portion 133. In some embodiments, the sixth and seventh angles a₆, a₇, may range from about 10-125°, individually. In some embodiments, the sixth and seventh angles a₆, a₇ may range from about 30-105°, individually. In some embodiments, the sixth and seventh angles a₆, a₇ may range from about 40-95°, individually. In some embodiments, the sixth and seventh angles a₆, a₇ may each be generally about 90°. In some embodiments, the sixth and seventh angles a₆, a₇ may each be generally about 45°.

As further shown in FIGS. 16A-16B, the first distal portion 133 (of the first side member 130) is spaced distally and vertically from the second arm 120 by the first offset portion 132. The first distal portion 133 is coupled to the first jaw member 160. In some embodiments, the first distal portion 133 is non-pivotably coupled to the first jaw member 160.

As shown in FIGS. 16A and 16C, the second side member 140 includes a second offset portion 142 positioned between a generally planar second proximal portion 141 and a generally planar second distal portion 143. The second proximal portion 141 defines a longitudinal axis A₁₃ and the second distal portion 143 defines a longitudinal axis A₁₄ offset from the longitudinal axis A₁₃ of the second proximal portion 141. The longitudinal axes A₁₃, A₁₄ being generally parallel to each other.

The second offset portion 142 of the second side member 140 defines a transverse axis T₄ extending at an eighth angle a₈ relative to the longitudinal axis A₁₃ of the second proximal portion 141 and/or extending at a ninth angle a₉ relative to the longitudinal axis A₁₄ of the second distal portion 143. In some embodiments, the eighth and ninth angles a₈, a₉ may range from about 10-125°, individually. In some embodiments, the eighth and ninth angles a₈, a₉ may range from about 30-105°, individually. In some embodiments, the eighth and ninth angles a₈, a₉ may range from about 40-95°, individually. In some embodiments, each of the eighth and ninth angles a₈, a₉ may be generally about 90°. In some embodiments, each of the eighth and ninth angles a₈, a₉ may be generally about 45°.

As further shown in FIGS. 16A and 16C, the first distal portion 143 (of the second side member 140) is spaced distally and vertically from the first arm 110 by the second offset portion 142. The second distal portion 143 is coupled to the second jaw member 165. In some embodiments, the second distal portion 143 is non-pivotably coupled to the second jaw member 165.

As best seen in FIGS. 16E and 17A in some embodiments, the first and second jaw members 160, 165 may be configured to be interchangeable and/or removable, as opposed to being affixed to the instrument 1000. By being interchangeable and/or removable, the same instrument 1000 can be used for a surgical procedure on both a left and right joint simply by switching the first and second jaw members 160, 165 from one side member to the other, as needed.

As depicted in FIGS. 17A and 17B, the first and second distal portions 133, 143 of the side members 130, 140, each include a member channel 136, 146 defined therethrough including leading to a locking aperture 137, 147. Each member channel 136, 146 being configured to receive and maintain a locking post 162, 167 of the jaw members 160, 165 therein. Each locking aperture 137, 147 configured to receive and maintain a deflectable locking member 164, 169 therein locking the jaw member 160, 165 into place.

As further depicted in FIGS. 17A and 17B, each jaw member 160, 165 may include a jaw body 161, 166 extending longitudinally between a locking post 162, 167 on a proximal end portion thereof and a shaped distal end portion 163, 168. Each jaw body 161, 166 defines a width greater than the respective locking post 162, 167. Each locking post 162, 167 is configured to fit and/or slide within the respective member channel 136, 146. The width of each jaw body 161, 166 is configured to match the width of the respective planar distal end portion 133, 143.

Each locking post 162, 167 includes one or more deflectable locking members 164, 169 configured to deflect inwardly to pass through the member channel 136, 146 and deflect outwardly when free of the member channel 136, 146 and particularly within the locking aperture 137, 147 thereby securing the jaw body 161, 166 to the respective distal portion 133, 143.

As depicted in at least FIGS. 16A, 16C, and 17A, in some embodiments, the first jaw member 160 may include a first shaped distal end portion 163 defining a longitudinal axis A₁₅ generally perpendicular to the longitudinal axis A₁₂ of the first distal portion 133 of the first side member 130 and/or the first and second jaw body 161, 166. The first shaped distal end portion 163 includes treads 179 to increase the surface area of the jaw member. In some embodiments, the treads 179 may extend generally parallel to the longitudinal axis A₁₂ of the first distal portion 133 of the first side member 130 and/or the first and second jaw body 161, 166.

As depicted in at least FIGS. 16A, 17A, and 19, in some embodiments, the second jaw member 165 may include a second shaped distal end portion 168 defining a longitudinal axis A₁₆ generally coplanar to the longitudinal axis A₁₄ of the second distal portion 143 of the second side member 140 and/or the second jaw body 166. The second shaped distal end portion 168 includes a proximal part 168a including at least one tine 178 and a distal part 168b. The second shaped distal end portion 168 (and/or the second jaw member 165) may define a greater length than the first shaped distal end portion 168 (and/or the first jaw member 160).

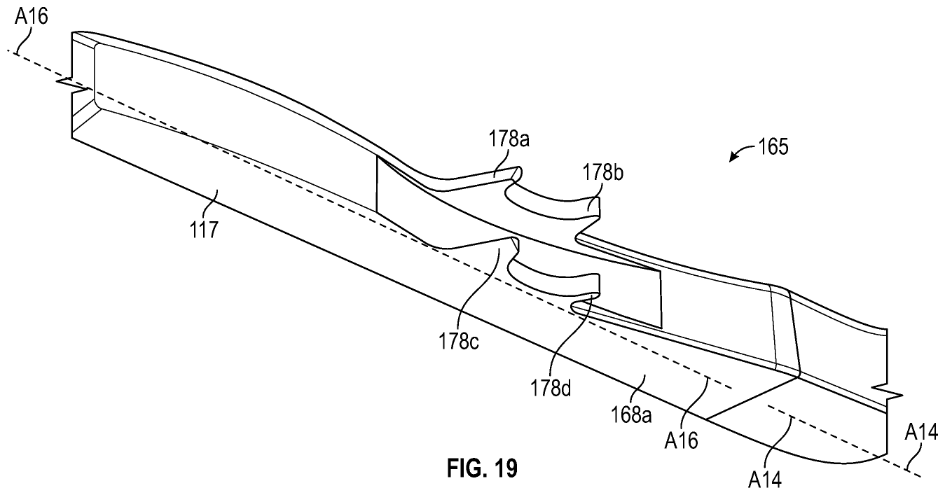

As shown in FIG. 19, the at least one tine 178 may be formed on an inner surface of a proximal part 168a of the shaped distal end portion 168 (and/or jaw member 165). In some embodiments, the shaped distal end portion 168 may include one or more pairs of tines 178a, 178b. In some embodiments, the shaped distal end portion 168 may include a first pair of tines 178a, 178b and a second pair of tines 178c, 178d on opposite edges of the jaw body 166.

In some embodiments, the first and second pair of tines 178a, 178b, 178c, 178d may be separated by a concave surface 195 on the proximal part 168a. The concave surface 195 providing additional space from the tips of each tine 178a-d to accommodate a jagged or crenulated outer surface of a bone graft.

A flange 177, and particularly a convex and/or rounded flange 177, is formed on a surface of the distal part 168b of the second shaped distal end portion 168. The proximal part 168a of the second shaped distal end portion 168, and particularly the tines 178a-d, is configured to align with the treads 179 of the first shaped distal end portion 163 in the closed configuration.

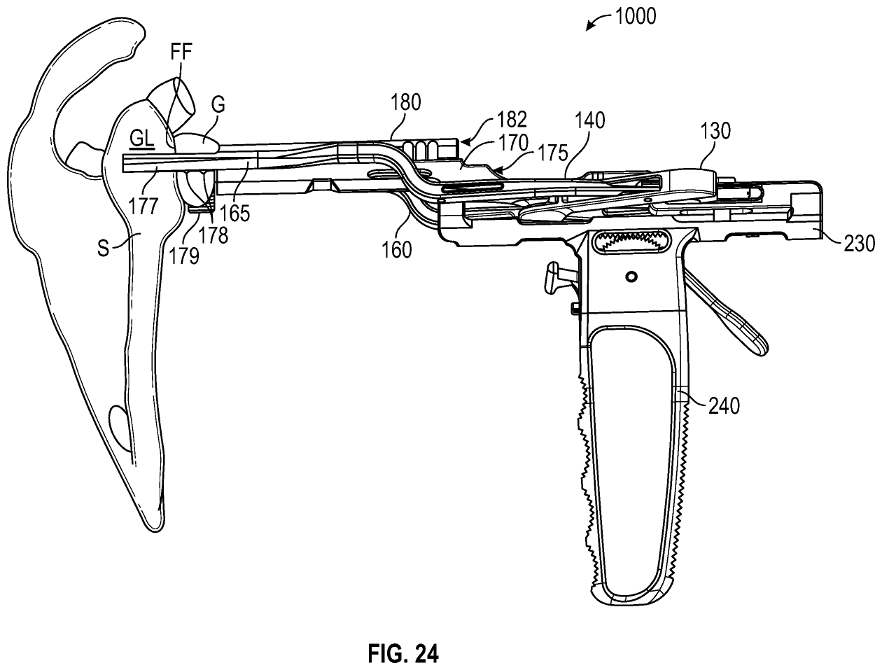

Since the flange and/or convex flange 177 extends beyond the one or more tines 178, the flange and/or convex flange 177 does not align with treads 179 and/or any portion of the first shaped distal end portion 163. The flange and/or convex flange 177 is designed to act as a guide for interacting with an outer side surface of a bone to which a graft may be secured to. For example, in a shoulder surgery such as a Latarjet procedure, the convex flange 177 is configured to fit onto the glenoid surface G2 of the scapula when the bone graft is properly positioned on a front surface of the scapula as best shown in FIG. 24.

The clamp assemblies of the surgical instruments 1000 including a pistol grip design as described herein may include any combination of the first and second jaw members described herein. Each of the jaw members may be configured to be affixed, removable, and/or interchangeable as needed.

The clamp assemblies 100 may further include a v-shaped linkage 150 pivotably connecting the first arm 110 to the second arm 120 proximal to the rotational joint 105. The v-shaped linkage 150 includes a first link member 151 pivotably coupled to a second link member 152 via a link pivot joint with the clamp pin 290 passing therethrough on a proximal end portion of each of the first and second link members 151, 152. Sliding of the clamp pin 290 in a proximal direction, via the gear assembly described herein, causes the first and second link members 151, 152 to narrow drawing the first and second arms 110, 120, the first and second side members 130, 140, and/or the first and second jaw members 160, 165 closers to each other (FIG. 16E). Sliding of the clamp pin 290 in a distal direction, via the gear assembly described herein, causes the first and second link members 151, 152 to separate forcing the first and second arms 110, 120, the first and second side members 130, 140, and/or the first and second jaw members 160, 165 away from each other (FIG. 17A).

A distal end portion of the first link member 151 is pivotably coupled to the first proximal pin 117 and received and maintained with the first channel 116. A distal end portion of the second link member 152 is pivotably coupled to the second proximal pin 127 and received and maintained with the second channel 126.

D. Guide Assembly of the Pistol Design

As further depicted in FIGS. 16A-17D, in some embodiments, the surgical instrument 1000 described herein may further include a guide assembly 170 extending in a distal direction along the support frame 230 and/or along a top of the cover 234 of the support frame 230. The guide assembly 170 may be further secured atop the rotational joint 105. However, unlike the first and second arms 110, 120, the guide assembly 170 does not pivot relative to the rotational joint 105. Rather the guide assembly 170 remains generally centered between the first and second jaw members 160, 165 in both the clamping and/or non-clamping configurations. The guide assembly 170 may include at least a guide carrier 174 including a fixed first bore 175 defined therethrough, and a removable guide insert 180 defining a second bore 182 therethrough. The removable guide insert 180 is configured to attach to and/or detach from the guide carrier 174.

The guide carrier 174 may be positioned along and/or affixed atop the cover 234 and distal the rotational joint 105. The carrier 174 extends between a first proximal part 174a and a second distal part 174b. The first proximal part 174a is affixed to the cover 234 distal the rotational joint 105 and the second distal part 174b extends distally away from the support frame 230 to a location prior to the first and second shaped distal end portions 163, 168 of the jaw members 160, 165. The guide carrier 174 may include at least a fixed first bore 175, a first guide shelf 176, a second guide shelf 177, and a guide locking aperture 178.

The fixed first bore 175 extends longitudinally through at least a portion of the guide carrier 174. In some embodiments, the fixed first bore 175 extends longitudinally through at least a portion of the first proximal part 174a and/or the second distal part 174b of the guide carrier 174. In some embodiments, the fixed first bore 175 extends longitudinally through at both the first proximal part 174a and the second distal part 174b of the guide carrier 174, either as a single bore or a plurality of bores spaced intermittently along the length of the carrier 174.

The first guide shelf 176, the second guide shelf 177, and the guide locking aperture 178 are located on top of the fixed first bore 175 of the carrier 174. The first guide shelf 176, the second guide shelf 177, and the guide locking aperture 178 being configured to receive, maintain, and/or support the removable guide insert 180 defining a second bore 182 therethrough.

The first guide shelf 176 extends distally along a first plane of the first proximal part 174a of the carrier guide 174. The first guide shelf 176 leads to the guide locking aperture 178 at a distal end thereof. The second guide shelf 177 extends distally along a second plane of the second distal part 174b of the carrier guide 174, the second plane being different than the first plane of the first guide shelf 176. The second guide shelf 177 located over the guide locking aperture 178.

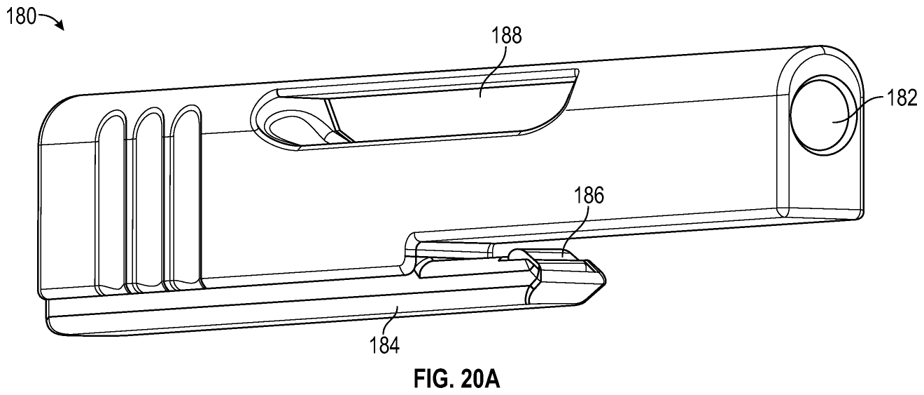

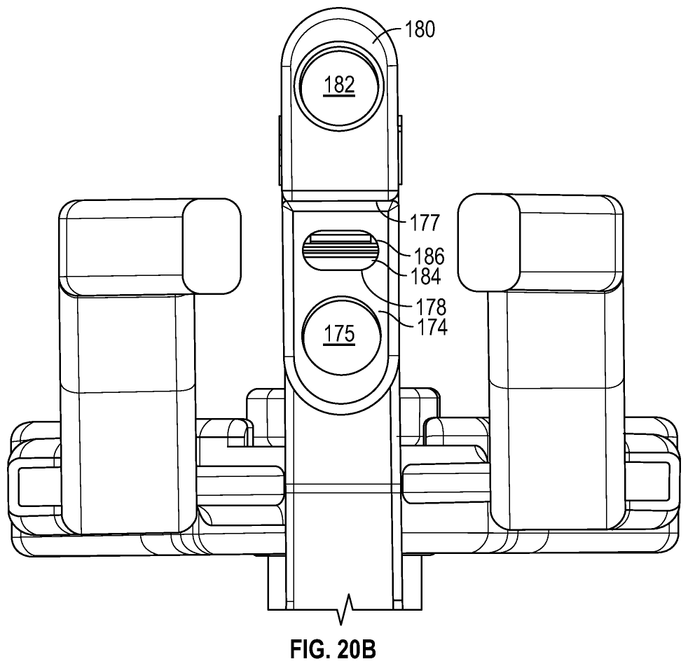

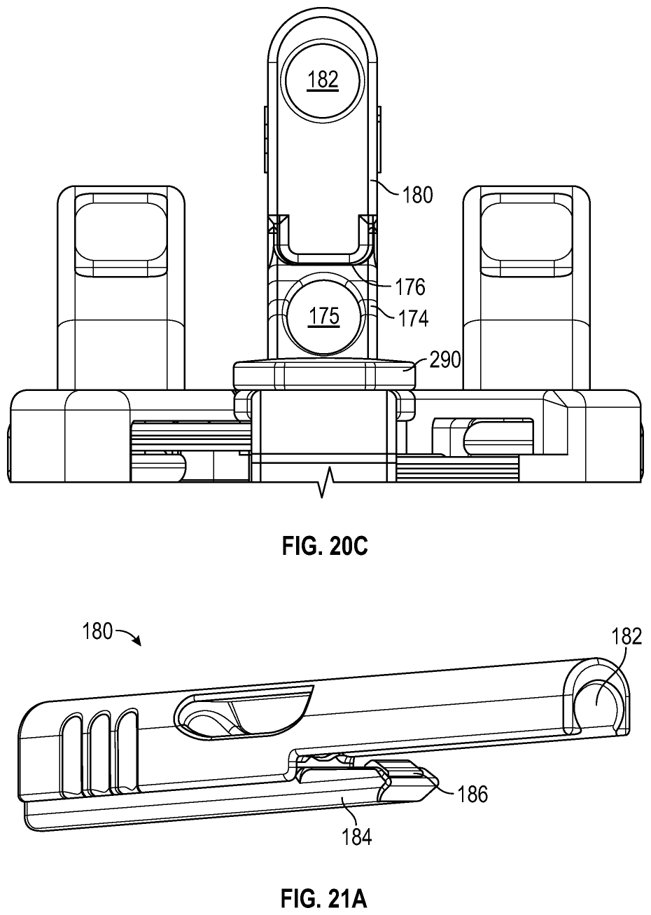

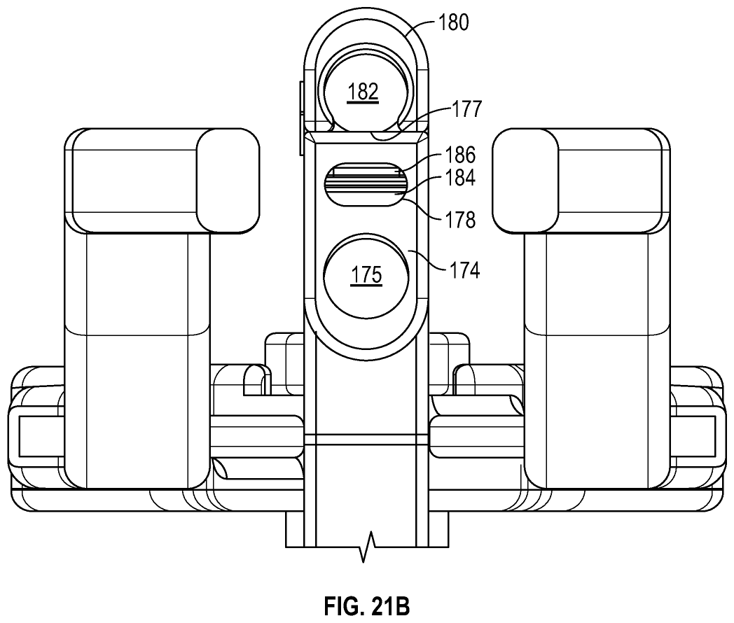

As shown in at least FIGS. 20A-21B, the removable guide inserts 180 described herein include at least a second bore 182 defined therethrough with guide locking post 184 located beneath the second bore 182. The guide locking post 184 including a deflectable guide locking member 186 located on top of a distal part thereof. The guide locking post 184 and deflectable guide locking member 186 configured to be received and/or slid within the guide locking aperture 178 to secure the guide insert 180 to the guide carrier 174. To remove or interchange the guide insert 180 from the carrier 174, the deflectable guide locking member 186 can be deflected and/or pressed into the guide locking post 184 and slid proximally away from the guide carrier 174.

The first fixed bore 175 and the second bore 182 may have a diameter (although not intended to indicate only circular openings) ranging between about 2 and 6 mms, and in some embodiments from about 3 to 5 mms, and in particular embodiments being about 4 mms. The first and second bores 175, 182 may have the same or different size diameters.

In some embodiments, the guide assembly 170 may further include a guide channel 172. The guide channel 172 may be positioned along and/or affixed atop the cover 234 and distal the pin slit 238. The guide channel 172 may extend longitudinally between the pin slit 238 and the rotational joint 105. The channel guide 172 may be designed to provide initial guidance for a drill bit and/or fastener (e.g., pins, screws, bolts, nails, and the like) therethrough prior to entering the fixed first bore 175 aligned therewith and spaced distally therefrom.

In some embodiments, the guide channel 172 may be an open guide channel as shown. It is further envisioned that in embodiments, the guide channel may alternatively be a closed guide channel.

In some embodiments, the bottom surface of the guide locking post 184, opposite the deflectable guide locking member 186, may be shaped to match and/or mate with the shape of the first guide shelf 176.

In some embodiments, the guide insert 180 may include a guide window 188 designed to provide access and/or visibility to at least a portion of the second bore 182 defined within a middle portion of the insert 180.

In some embodiments, the guide assembly 170 is configured to receive interchangeable guide inserts 180 including a second bore 182 defined therein at different heights. For example, as shown in FIGS. 21A-21B, in some embodiments, the guide insert 180 includes a second bore 182 configured to rest directly on the second guide shelf 177. In such embodiments, the first and second bores 175, 182 are as close as possible while remaining vertically aligned on the carrier 174. Since the location of the first bore 175 is predetermined and/or fixed on the guide carrier 174, the space between the first and second bores 175, 182, can be predetermined and/or adjusted by interchanging guide inserts 180 including second bores 182 of various heights. For example, as shown in FIGS. 5A-6B, the second bore 182 is designed to be spaced a certain distance from both the second guide shelf 177 and/or the first fixed bore 175.

In some embodiments, the first and second bores 175, 182 may be separated by a distance ranging from about 5 to 18 mms, and in some embodiments from about 9 to 15 mms.

In some embodiments, the guide assembly 170 of the pistol design may be part of the handle portion of the instrument. In some embodiments, the guide assembly may be configured to be removably attached to the instrument.

Each of the guide assemblies as described herein are interchangeable with each of the clamp designs. For example, in some embodiment, the surgical forceps design (at least FIGS. 1A-1C) may include the modular guide assembly described previously with regard to the surgical clamp pistol design (at least FIGS. 17A-17D), and more particularly, the modular guide assembly may include a guide carrier extending from a rotational joint of the forceps design, the guide carrier configured to receive one or more removable guide inserts as described herein. In another example, in some embodiments, the surgical clamp pistol design (at least FIGS. 17A-17D) may include the non-modular guide assembly described previously with regard to the surgical forceps design (at least FIGS. 1A-1C), and more particularly, the non-modular guide assembly may include a guide wall positioned on and extending distally from a rotational joint.

In some embodiments, the guide assembly may not be part of the surgical instrument. Rather, the guide assembly may be part of a separate tool designed to be positioned distal to the handle portion of the surgical instruments described herein and proximal the jaw members.

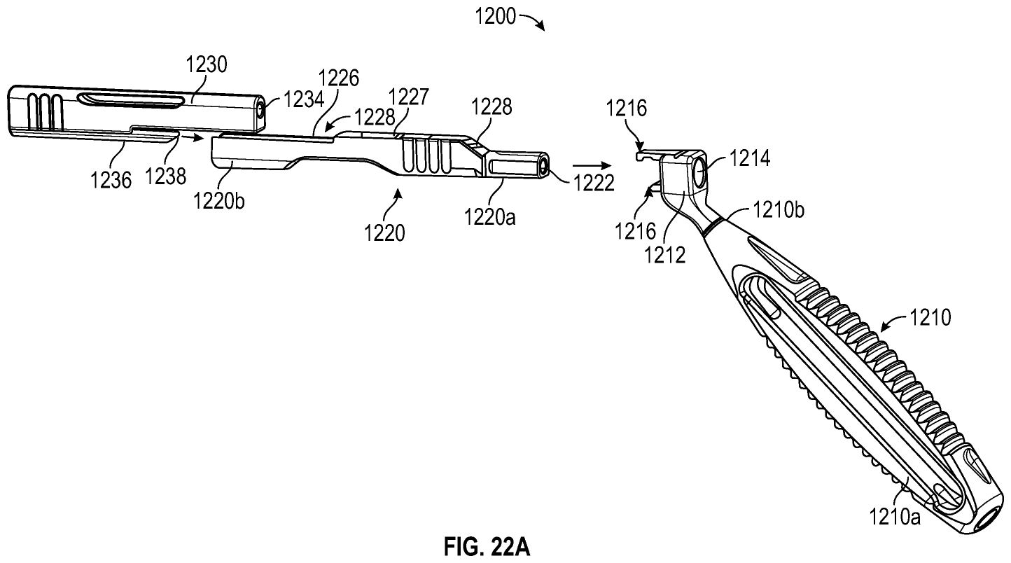

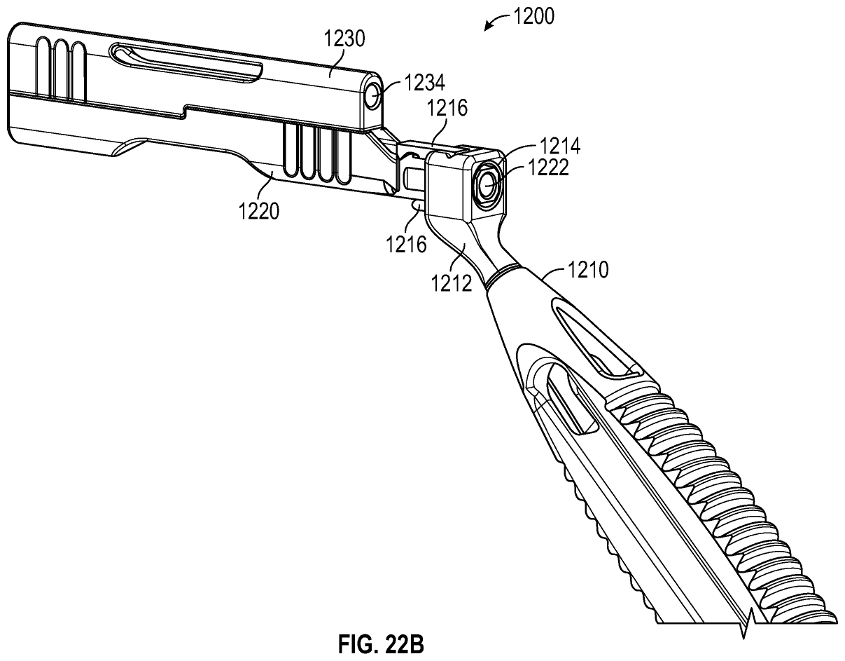

As shown in FIGS. 22A and 22B, a separate positioning tool 1200 can be formed by combining a tool handle 1210, with a guide base 1220 and a removable guide insert 1230. The tool handle 1210 includes a first proximal part 1210a configured to be held by a user and a second distal part 1210b including a working head 1212 offset from the longitudinal axis of the handle 1210.

The working head 1212 includes an anchoring hole 1214 defined therethrough and one or more locking brackets 1216 extending distally therefrom. The anchoring hole 1214 sized for the receipt of a proximal end part 1220a of the guide base 1220 including a first bore 1222 defined therethrough. A portion of the proximal end part 1220a of the guide base 1220 being configured to also snap into a locked position between the locking brackets 1216 when slid into the anchoring hole 1214.

The guide base 1220, similar to the guide carrier 174, further includes a first guide shelf 1226, a second guide shelf 1227, and a guide locking aperture 1228. However, the lower first guide shelf 1226 is located distal to both the guide locking aperture 1228 and the higher second guide shelf 1227. In this configuration, the removable guide insert 1230 is configured to be slid proximally onto the guide base 1220, and particularly the first guide shelf 1226 into the guide locking aperture 1227, to secure the insert 1230 to the base 1220.

As further shown in FIGS. 22A-22B, the removable guide inserts 1230 described herein include a second bore 1234 defined therethrough and with a guide locking post 1236 located beneath the second bore 1234. The guide locking post 1236 including a deflectable guide locking member 1238 located on top of a distal part thereof. The guide locking post 1236 and deflectable guide locking member 1238 configured to be received and/or slid within the guide locking aperture 1227 to secure the guide insert 1230 to the removable guide base 1220. To remove or interchange the guide insert 1230 from the base 1220, the deflectable guide locking member 1238 can be deflected and/or pressed into the guide locking post 1236 and slid distally away from the guide base 1220.

The combination of the tool handle 1210, the guide base 1220 and the removable guide insert 1230 form a positioning tool 1200 including vertically aligned first and second bores 1222, 1234 for the passage and guidance of bone drill bits and/or surgical fasteners therethrough. In some embodiments, the separate positioning tool 1200 is configured for determining where to drill and/or fasten a bone graft on to the scapula in an orthopedic surgical procedure, such as Latarjet procedure.

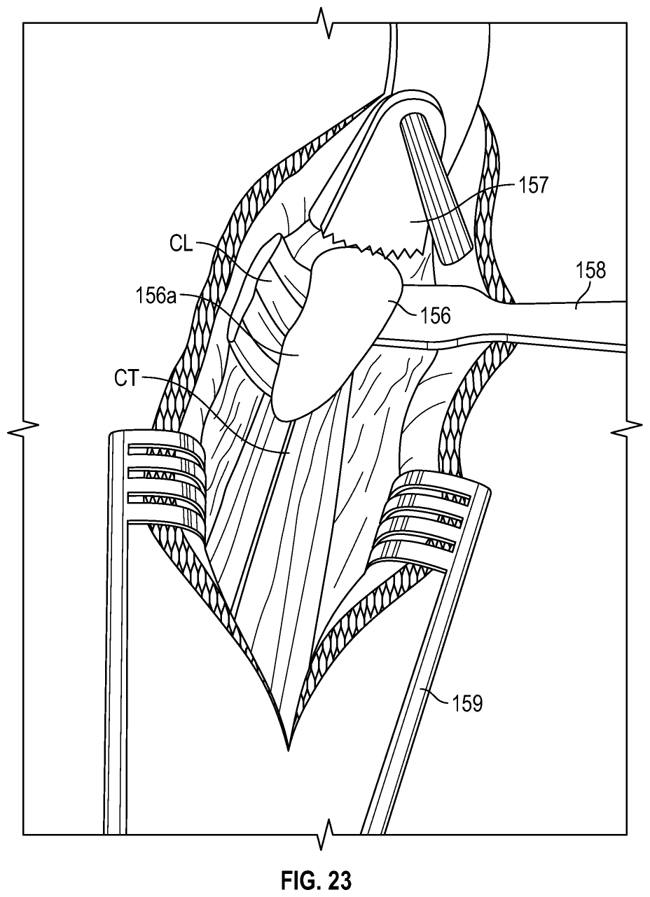

FIGS. 23-25B illustrate a plurality of steps of a surgical procedure such as a Latarjet procedure carried out by using a surgical instrument 1000 in a pistol configuration as described herein. As explained above, one benefit of the novel features of the instrument is that of carrying out a method which is remarkable in that it consists of positioning by means of a surgical instrument a bone graft on the scapula and drilling and/or fastening said bone graft at the same time as the scapula with the aid of a guide assembly including one or more bores and a surgical clamping instrument described herein including an ergonomic pistol-type grip in order to position one hand of the user beneath the instrument out of the path of any drilling and/or fastening performed above the grip.

As illustrated in FIG. 23, a pair of retractors 159 maintain access to the coracoid process 156 for cutting with a bone-cutting device 157, such as a sagittal saw equipped with a 90 degree blade. A periosteal elevator 158 may be used to expose the knee of the coracoid process 156. A coracoacromial ligament stump CL remains attached to the coracoid process 156 (and/or the bone graft G).

The surgical instruments 1000 described herein may be used before, during, and/or after the cutting of the coracoid process 356 to clamp or grasp the coracoid graft G in a generally fixed position. It is envisioned that the instrument 1000 will approach the coracoid process 356 straight on with a superior face 356a of the coracoid 356 positioned between with the first and second jaw members 160, 165 facing the bores 175 and 182 of the instrument 1000. In this configuration, the bone graft G may not need to be further manipulated and/or rotated prior to reattachment to the scapula S.

FIG. 24 illustrates the position of coracoid or bone graft G within the jaw members 160, 165 of the surgical instrument 1000 described herein. The bone graft G may include the coracoacromial ligament stump and remain attached to the conjoined tendon. As noted hereinabove, the combination of the tines 178 and the concave surface of the second jaw member 165 creates extra and/or sufficient space therebetween for the coracoacromial ligament stump to be positioned without damage, while the graft G is secured between the treads 179 on the first jaw member 160 and the tines 178 on the second jaw member 165. In this position, a majority of the bone graft G is accessibly for resurfacing, if needed.

Alternatively, in some instances, the bone graft G may also be rotated and/or repositioned within the instrument 1000 to be resurfaced. To do this, the bone graft G is turned in the instrument to have more access to the surface to be freshened.

As further illustrated in FIG. 24, to secure the bone graft G to the anterior or front face FF of the scapula S, the graft G may be locked between the jaw members 160, 165 with the convex flange 177 positioned bearing against the generally concave glenoid joint surface GL of the scapula S. When the convex flange 177 is properly seated in the glenoid joint GL, the proper placement of the bone graft G on the front face FF of the scapula S is likely, if not a certainty.

When the instrument 10 positions the bone graft G on the front face FF of the scapula S, the guide assembly 170, and particularly the first and second bores 175, 182 are centrally aligned and/or centered on the front face of the bone graft G. A drill bit (not shown) can be passed through either or both of the bores 175, 182 to drill holes through the graft G and also the scapula S.

After producing the drill holes, the instrument 1000 may be removed while the drill bits are retained in the drill holes. The drill bits may then be successively removed and replaced with any suitable fastener, such as a compression screw or a loop in suitable flexible material.

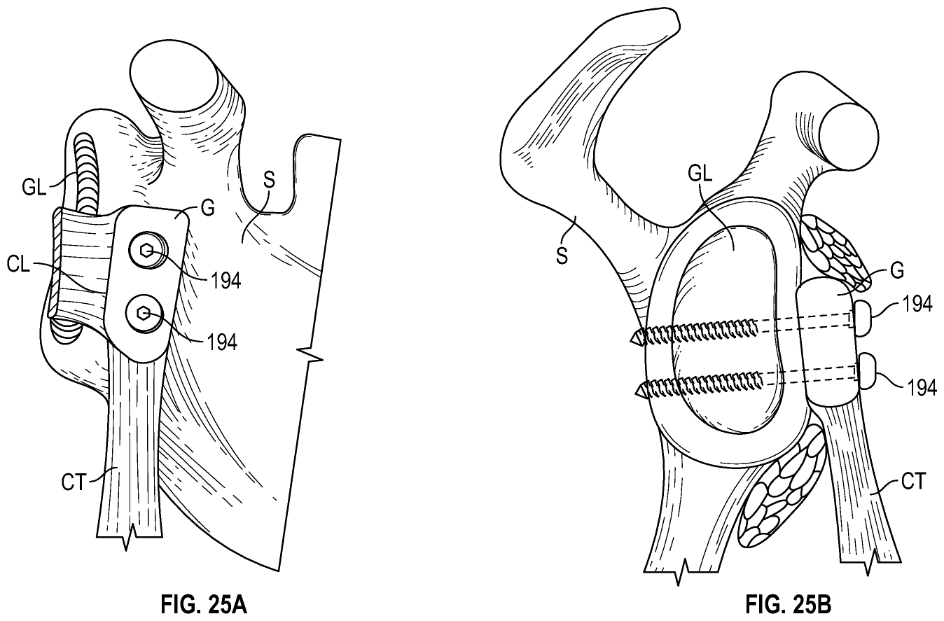

As shown in FIGS. 25A and 25B, two fasteners 194, such as compression screws, affix the bone graft G to the scapula S with the conjoined tendon CT intact and the coracoacromial ligament CL extending therefrom and ready to be reattached.

Although FIGS. 24-25B depict the use of the surgical instruments 1000 described herein on an anterior face of a shoulder bone, such as the scapula, the surgical instruments described herein may also be configured to be used along a posterior face of a shoulder bone, such as the scapula, as needed. For example, in some embodiments, the surgical instruments described herein may be used in a posterior instability to position and fix a posterior graft in a similar manner as shown for an anterior graft, only on a posterior site. In addition, the surgical instruments described herein are not intended to be limited only to shoulder surgery, but rather may be designed to be useful in a variety of different surgical procedures, including but not limited to orthopedic surgeries and/or open surgical procedures.

Surgical kits are also provided herein including any of the surgical instruments described herein and one or more of the removable guide inserts as described herein. The one or more of the removable guide inserts being of different sizes and/or dimensions. The surgical kits may further include a separate surgical tool handle including a removable guide base. The surgical kits may further include one or more removable jaw members of various configurations.

In some embodiments, the surgical kits may include any of the surgical instruments described herein and one or more removable jaw members of various configurations.

It will be understood that various modifications may be made to the embodiments disclosed herein. Thus, those skilled in the art will envision other modifications within the scope and spirit of the disclosure.

What is claimed is:

1. A surgical instrument including a clamp assembly comprising:
a first arm having a first proximal end portion and a first distal end portion opposite the first proximal end portion, the first arm including a first proximal pivot joint and a first distal pivot joint,
a second arm having a second proximal end portion and a second distal end portion opposite the second proximal end portion, the second arm including a second proximal pivot joint and a second distal pivot joint, wherein the first and second arms are pivotably coupled to each other via a rotational joint,
a first and second side member positioned on opposite sides of the rotational joint,
a first and second jaw member,
wherein
the first side member includes a first offset portion positioned between a first proximal planar portion and a first distal planar portion, a first proximal end portion of the first proximal planar portion pivotably coupled to the first arm via the first proximal pivot joint and a first distal end portion of the first proximal planar portion pivotably coupled to the second arm via the second distal pivot joint,
the second side member includes a second offset portion positioned between a second proximal planar portion and a second distal planar portion, a first proximal end portion of the second proximal planar portion pivotably coupled to the second arm via the second proximal pivot joint and a second distal end portion of the second proximal planar portion pivotably coupled to the first arm via the first distal pivot joint.

2. The surgical instrument of claim 1, wherein the clamp assembly further comprises a v-shaped linkage including a first link member pivotably coupled to a second link member via a link pivot joint.

3. The surgical instrument of claim 2, wherein
a distal end portion of the first link member is pivotably coupled to the first proximal pivot joint and received and maintained in a first channel defined within the first arm and
a distal end portion of the second link member is pivotably coupled to the second proximal pivot joint and received and maintained in a second channel defined within the second arm.

4. The surgical instrument of claim 3, wherein
the first proximal planar portion and the first distal planar portion are spaced vertically by the first offset portion,
the second proximal planar portion and the second distal planar portion are spaced vertically by the second offset portion,
the first and second proximal planar portion are coplanar in a first plane, and the first and second distal planar portion are coplanar in a second plane.

5. The surgical instrument of claim 4, further comprising a guide assembly coupled to the rotational joint, the guide assembly including one or more guide walls extending vertically away from the rotational joint, the guide wall including one or more bores defined therethrough, the bores configured to receive drill bits or fasteners therethrough.

6. The surgical instrument of claim 5, wherein the guide assembly further comprises a pair of guide arms positioned on a proximal end portion of the guide assembly, the guide arms separated by a guide slit configured to receive and maintain a guide post therein, the guide post extending from the link pivot joint of the v-shaped linkage.

7. The surgical instrument of claim 5, wherein the one or more bores includes one bore which is in the second plane defined by the first and second distal planar portion of the first and second side member.

8. The surgical instrument of claim 5, wherein the one or more bores includes two bores which are spaced vertically by the second plane defined by the first and second distal planar portion of the first and second side member.

9. The surgical instrument of claim 4, further comprising a guide assembly including a guide carrier including a fixed first bore defined therethrough, and a removable guide insert including a removable second bore defined therethrough.

10. The surgical instrument of claim 9, wherein the guide carrier extends between a proximal part and a distal part, the proximal part affixed atop the rotational joint and the distal part extending away in a distal direction to a location prior to the first and second jaw members, the guide carrier further comprising a first guide shelf defining a first plane proximal a second guide shelf defining a second plane different than the first plane, the first guide shelf leading to a guide locking aperture configured to receive a guide locking post of the removable guide insert.

11. The surgical instrument of claim 10, wherein the removable guide insert further comprises the guide locking post located beneath the second bore, the guide locking post further comprising a deflectable guide locking member on top of a distal part thereof, the guide locking post and the deflectable guide locking member configured to be received within the guide locking aperture to secure the removable guide insert to the guide carrier.

12. The surgical instrument of claim 1, wherein:
the first jaw member is removably coupled to the distal planar portion of one of the first or second side members, and
the second jaw member is removably coupled to the distal planar portion of one of the first or second side members different than the first jaw member.

13. The surgical instrument of claim 12, wherein:
the first jaw member includes a first jaw body having a concave inner surface including longitudinal treads on a distal end portion of the jaw body and
the second jaw member includes a second jaw body having an inner surface including one or more tines with a convex flange extending distally beyond the tines and not longitudinally aligned with the tines.

14. The surgical instrument of claim 1, wherein a proximal end portion of the first arm further comprises a first passage defined therein behind a deflectable wall defined on an inner surface of the first arm.

15. The surgical instrument of claim 14, further comprising a ratchet assembly including a shaped first end portion pivotably coupled to a collar on the first proximal end portion of the first arm, the shaped first end portion including a first rib, a second rib, and a locking arm extending therefrom, wherein the first rib extends into the first passage behind the deflectable wall, the second rib extends away from the first rib, and the locking arm includes a plurality of teeth on at least one surface thereof, at least one of the teeth configured to engage a pawl positioned on a second proximal end portion of the second arm to lock the first and second arms in a fixed position relative to each other.

16. The surgical instrument of claim 2 further comprising:
a handle portion including a support frame positioned on top of a handle body, the support frame configured to support the clamp assembly,
a gear assembly operably coupling the handle portion to clamp assembly, and
a guide assembly secured to the support frame.

17. The surgical instrument of claim 16, wherein the handle body extends a first longitudinal axis between a first free end portion and a second end portion connected to the support frame, and the second end portion of the handle body defines a handle cavity therein, the handle cavity configured to receive a first lower portion of the gear assembly therein, the first lower portion of the gear assembly including a first gear, a trigger, and a locking member.

18. The surgical instrument of claim 17, wherein:
the first gear is configured to rotate about a pivot point inside the handle cavity and includes teeth along an outer perimeter thereof, and
the trigger is affixed to the first gear by a trigger arm, the trigger arm extending from the first gear inside the handle cavity, through a proximal handle slot defined on a proximal face of the handle body, to the trigger positioned outside the handle cavity, the trigger arm configured to pivot within the proximal handle slot to cause the first gear to pivot inside the handle cavity.
the locking member includes a locking arm, a locking pawl, and a tab member, the locking arm extending between a first fixed end portion secured to a portion of the handle portion and a second free end portion including the locking pawl and the tab member on opposite sides thereof, the locking arm positioned within and extending along a length of a distal handle slot defined in a distal face of the handle body, the tab member extending out of the distal handle slot and the locking pawl configured to interact with the teeth of the first gear remain inside the handle cavity.

19. The surgical instrument of claim 18, wherein the gear assembly further comprises a second upper portion received and/or maintained within the support frame, the second upper portion of the gear assembly including a second gear, a biasing member, and a clamp pin connecting the gear assembly to the clamp assembly.

20. The surgical instrument of claim 19, wherein:
the second gear is a linear gear including a second set of teeth configured to interact with a portion of the first set of teeth of the first gear, the second gear located within a gear channel of a base of the support frame, the second gear configured to slide side-to-side, relative to the first gear, within the gear channel.
the biasing member is positioned proximal the second gear within the base of the support frame, the biasing member configured to force the second gear to slide distally within the gear channel.
the clamping pin extends between a proximal end portion of the second gear to a pin slit defined in a cover of the support frame, with a proximal part of the v-shaped linkage positioned therebetween, the clamping pin configured to slide side-to-side within the pin slit forcing the proximal part of the v-shaped linkage to move proximally or distally thereby forcing the jaw members to open or close, respectively.
